(19) United States
(12) Patent Application Publication
(10) Pub. No.: US 2006/0099713 A1
(43) Pub. Date: May 11, 2006

(54) Targeted-assisted iterative screening (tais):a novel screening format for large molecular repertoires

(75) Inventors: Alexei Kourakine, Novato, CA (US); Dale Bredesen, Novato, CA (US)

Correspondence Address: QUINE INTELLECTUAL PROPERTY LAW GROUP, P.C., P O BOX 458, ALAMEDA, CA, 94501, US

(73) Assignee: BUCK INSTITUTE

(21) Appl. No.: 10/515,210

(22) PCT Filed: Oct. 1, 2002
(86) PCT No.: PCT/US02/31349

§ 371 Date: Nov. 25, 2005

(51) Int. Cl. G01N 33/00

(52) U.S. Cl. 436/86

(57) ABSTRACT

This invention provides a new in vitro screening method for the detection of protein-protein and other interactions. The method has been developed and applied to a commercial cDNA library to search for novel protein-protein interactions. PDZ, WW and SH3 domains from PSD95, Nedd4, Src, Abl and Crk proteins were used as test targets. 12 novel putative and 2 previously reported interactions were identified for 6 protein interaction modules in test screens. The novel screening format, dubbed TAIS (target-assisted iterative screening), provides an alternative platform to existing technologies for a pair-wise characterization of protein-protein, and other, interactions.

### CROSS-REFERENCE TO RELATED APPLICATIONS

This application claims priority to and benefit of U.S. Ser. No. 60/326,566, filed on Oct. 1, 2001, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made, in part, with Government Support under Grant No: NS33376 awarded by the National Institutes of Health. The Government of the United States of America may have certain rights in this invention.

### FIELD OF THE INVENTION

This invention pertains to the field of proteomics. In particular, this invention pertains to a dual screening method for determining interactions between members of a library and various targets that allows simultaneous screening for large numbers of interactions (e.g. protein-protein interactions) between library members and the target(s).

### BACKGROUND OF THE INVENTION

Understanding the cell at a system level involves a comprehensive analysis of both the structure and the dynamics of cellular protein interaction networks. A large-scale analysis of protein-protein interactions has been attempted in lower eukaryotes, providing a first glimpse of the astounding structural complexity of the protein interaction webs (Walhout et al. (2000) Science 287: 116-122; Uetz et al. (2000) Nature 403: 623-627; Ito et al. (2001) Proc. Natl. Acad. Sci., USA, 98: 4569-4574).

Concurrently, a completed draft of the human genome has now delineated the dimensions of the human proteome (Venter et al. (2001) Science 291: 1304-1351; Lander et al. (2001) Nature 409: 860-921). Assembling of the estimated 30,000 to 50,000 human gene products into a comprehensive protein interaction map would provide a view of the cell as a molecular system or molecular network and provide a system in which the timing and dynamics of protein-protein and other interaction events, could be examined.

Currently, the only practical method for a pair-wise characterization of protein-protein interactions with relatively high throughput is the yeast two hybrid system (Fields and Song (1989) Nature 340: 245-246). However, a high rate of false positives, poor performance in case of transcription factors, membrane bound, mistargeted and toxic proteins limit applicability of the two-hybrid system.

The limitations of the two-hybrid system have been recently highlighted by results of independent large scale protein interaction experiments performed on the yeast proteome ((Ito et al. (2001) Proc Natl Acad Sci USA 98: 4569-74; Uetz et al., (2000) Nature 403: 623-627). The comparison revealed unexpectedly low overlap between the results of two groups (about 20%). Moreover, analysis of protein-protein interactions deposited in the Yeast Proteome Database showed that systematic two-hybrid projects failed to reproduce as much as approximately 90% of the interactions identified in conventional two-hybrid screens (Ito et al. (2001) Proc Natl Acad Sci USA 98: 45694574).

The absence of a positive control in two-hybrid systems is particularly problematic as this approach is known for its abundance of false positives. In addition, it is known that the two-hybrid system is poorly designed for the identification of proteins; interacting with transcription factors, and with toxic, membrane-bound, mistargeted or large proteins.

Therefore, the development of new methods with high throughput potential to characterize protein-protein interactions is of paramount importance, and increasingly so with the increasing availability of the human, and other, genome sequences.

### SUMMARY OF THE INVENTION

The present invention pertains to a novel, rapid in vitro screening method for the identification and characterization of protein-protein interactions (e.g. interactions mediated by specialized protein modules such as SH3, PDZ and WW domains). The method is well suited to large-scale functional genomics approaches. In essence the present method combines the advantages of phage display technology and cDNA expression libraries.

In one embodiment, this invention provides a method of identifying interacting proteins from a plurality of potentially interacting proteins. The method typically involves i) contacting one or more targets (e.g. target proteins) with a protein display library comprising a plurality of potential binding proteins for the one or more target proteins; ii) selecting members of the protein display library that bind to the one or more target proteins to provide a preselected set of potential binding proteins; iii) separating the members of the preselected set of potential binding proteins from the bound target protein and localizing and/or immobilizing the members on a solid support such that the members are spatially addressable; and iv) contacting members of the preselected set of potential binding proteins with one or more target proteins; and v) detecting binding of members of the preselected set of potential binding proteins with the one or more target proteins whereby binding of a member of said set of potential binding partners with a target protein indicates that the member and the target protein are interacting proteins.

In certain preferred embodiments, the target proteins are attached to a solid support during the first contacting step. The protein display library can be any convenient display library. Preferred display libraries include, but are not limited to phage display, bacterial display, yeast display, eukaryotic virus display library, direct plasmid display library, and so forth. In certain embodiments, the library is an in vitro display library (e.g. covalent display technology (CDT), polysome display, eukaryotic in vitro transcription/translation systems, RNA-peptide fusions, and the like). Such libraries typically comprise at least 100 different members, preferably at least 1000 different members, more preferably at least 10,000 and most preferably at least 10⁶, 10⁷, 10⁸, 10⁹ or 10¹⁰ different members. In particularly preferred embodiments, the library displays a cDNA library (e.g. from a particular organism, tissue, cell type, etc.).

In certain embodiments, amplification of preselected subset of potential interactors of the target(s) is often performed, and can be performed in a spatially addressable manner. Thus, in certain embodiments, the “separating” comprises amplifying members of the protein display library that bind to said one or more target proteins and/or the separating and/or immobilizing comprises amplifying members of the protein display library that bind to said one or more target proteins. The amplifying can comprise amplification of the members when they are spatially separated and addressable.

In certain embodiments, the selecting comprises removing unbound members of the display library from the solid support. The selecting can comprise capturing one or more target proteins and/or bound library members (i.e. in a bound complex) using an affinity matrix. In certain embodiments, contacting members of the preselected set of potential binding partners with one or more target proteins comprises adsorbing members of the preselected set of potential binding partners to a solid support (e.g. a membrane). The detecting can be by means of a label attached to the target protein(s). Preferred labels include, but are not limited to a fluorescent label, a radioactive label, an enzymatic label, a colorimetric label, and a magnetic label.

In certain preferred embodiments, the contacting of step (i) comprises contacting the one or more target proteins with a protein display library where said one or more target proteins are attached to a solid support; the contacting of step (iv) comprises attaching members of the preselected set of potential binding proteins to a solid support to provide a set of attached preselected potential binding proteins and contacting the attached preselected potential binding proteins with the one or more target(s) (e.g. target proteins). The target proteins used in the contacting of step (iv) can be labeled with a detectable label before, during, or after the target proteins are contacted to the preselected potential binding proteins. In certain embodiments, the method further comprises sequencing the nucleic acid encoding the displayed protein on a member of the preselected display library that binds to the target protein. In certain embodiments, the contacting of step (i) comprises contacting one or more target proteins with a protein display library where said one or more target proteins and the protein display library are in solution. The selecting step can comprise capturing target proteins bound to members of the protein display library using an affinity matrix that specifically binds the target proteins or a tag attached to the target proteins. The contacting of step (iv) can comprise attaching members of said preselected set of potential binding proteins to a solid support to provide a set of attached preselected potential binding proteins and contacting the attached preselected potential binding proteins with the one or more target proteins. In certain preferred embodiments, the detecting comprises determining the amino acid sequence of a member of the set of potential binding partners (e.g., binding proteins) that binds a target protein. The method can further involve recording the amino acid sequence or identity of a member of the set of potential binding partners that binds a target protein in a database of proteins that interact with the target.

The methods described herein are not limited simply to target protein(s). Essentially any target moiety can be used. Such moieties include, but are not limited to various natural or synthetic chemical compounds including, but not limited to drugs, small organic molecules, nucleic acids, proteins, glycoproteins, carbohydrates, and the like. Similarly, the display library need not be limited to proteins. Virtually any moiety that can be displayed in a library is suitable. Particularly preferred display libraries include, but are not limited to protein or nucleic acid display libraries.

In one particularly preferred embodiment, this invention provides a method of identifying proteins or nucleic acids that interact with target moieties from a nucleic acid or protein library comprising a plurality of nucleic acids or proteins. The method typically comprises, i) contacting one or more target moieties with the library; ii) selecting members of the library that bind to the one or more target moieties to provide a preselected set of potential binding partners; iii) separating the members of the preselected set of potential binding partners from the bound target and immobilizing the members on a solid support such that the members are spatially addressable; iv) contacting members of the preselected set of potential binding partners with one or more target moieties; and v) detecting binding of members of the set of potential binding partners with said one or more target moieties whereby binding of a member of the set of potential binding partners with a target binding moiety indicates that said member is a binding partner that interacts with the target moiety. Preferred libraries include, but are not limited to a phage display library, a bacterial display library, a yeast display library, a eukaryotic virus library, a direct encoded plasmid library, and the like. In certain preferred embodiments, the library is an in vitro display library (e.g. a covalent display technology (CDT) library, a polysome display library, an RNA-peptide fusion library, etc.). In certain embodiments, the target moiety is a nucleic acid (e.g. a DNA, an RNA), a lipid, a carbohydrate, a glycoprotein, or a small organic molecule.

This invention also provides a kit practicing any of the methods described herein. In one embodiment, the kit comprises a protein display library; and instructional materials providing protocols for the methods described herein.

Unlike traditional panning approaches that select for the best binders, TAIS eliminates the loss of weaker binders and propagation biases, that result from competition between individual phage during repetitive selection-amplification cycles. In addition, the method permits screening of significantly larger libraries than the ones routinely used in cDNA expression library screening. For example, if a practical limit of the cDNA expression library screening assay is 10⁶-10⁷ phage, the upper limit on the size of the library used in TAIS is defined by existing technologies of phage display library preparation, i.e., on the order of 10⁸-10¹² or more phage.

TAIS provides a number of advantages: The method does not require costly and sophisticated equipment, and can be used with commercially available reagents. The method involves only simple biochemical and microbiological manipulations, and, additionally because of the low cost is easily attainable for almost any lab, with minimal investment for setup. The method has a short turnaround time: normally within 24 hours an investigator will know whether or not a particular screen has been successful, and often, in 48 to 72 hours an investigator has DNA ready for sequencing to analyze the cDNAs selected in the screen. The screening is performed in vitro, i.e., under defined and manipulatable conditions; the readout is direct, and is easily accurately quantitated. The method provides a powerful tool to characterize ligand preferences of peptide recognition domains. In this application, cDNA libraries (e.g. phage-displayed cDNA libraries) have unique features when compared to traditional combinatorial peptide libraries. The lengths of the peptides in the library are not fixed. The libraries can feature natural peptide ligands of the target that provide internal references for physiologically relevant affinities and specificities of the interaction in question.

Since it is not usually known a priori within what length of the peptide ligand all determinants of a specific interaction reside and what are physiologically relevant interaction affinities, the features described above make displayed cDNA libraries an invaluable complement to traditional peptide libraries in the characterization of molecular recognition properties of peptide interaction modules.

Furthermore, TAIS allows the analysis of relatively weak and/or poorly propagating binders that are typically lost during the standard phage display panning procedure. Propagation biases and disparity in stabilities between different phages are of special issue in the case of cDNA libraries, since the size and composition of displayed polypeptides in such libraries vary greatly in comparison to more traditional peptide or antibody libraries.

We believe that the application of the screening format described here to cDNA libraries provides a powerful platform complementing existing technologies for a pair-wise characterization of protein-protein interactions. The relatively high efficiency and technical simplicity of the proposed screening method, as well as its readily standardized output, will allow TAIS to be utilized as a high throughput tool for mapping of protein-protein interactions.

Finally, it is noted that, in essence, the TAIS format allows efficient, target affinity-driven reduction of enormous molecular diversity in liquid phase to a manageable size sub-library immobilized in a spatially addressable form that can be processed robotically or manually. As such the screening method can be applied to a number of other large molecular diversities such as phage-displayed peptide and recombinant antibody libraries, cell displayed polypeptide libraries, etc. Iterative presentation of the target in two different molecular contexts facilitates minimization of non-specific interactions.

As indicated above, in preferred embodiments, the methods of this invention involve two screening steps. Generally the methods comprise: i) contacting one or more target proteins with a molecular library (e.g. a protein display library, nucleic acid display library) comprising a plurality of potential binding partners for the one or more targets (e.g. target proteins); ii) selecting members of the display library that bind to the one or more targets to provide a preselected set of potential binding partners; iii) separating said members of said preselected set of potential binding partners from the bound target and immobilizing said members on a solid support such that said members are spatially addressable; and iv) contacting members of the preselected (and optionally amplified) set of potential binding partners with one or more targets again; and v) detecting binding of members of the set of potential binding proteins with the one or more targets whereby binding of a member of the set of potential binding partners with a target indicates that the member and the target interact.

Contacting one or more Target Moieties with a Display Library.

In preferred embodiments, the methods of this invention typically involve an initial screen that entails contacting one or more target moieties with a library of potential binding partners (e.g. preferably nucleic acids or proteins). The library is preferably a display library, more preferably a protein display library (e.g. phage display, bacterial display, yeast display, eukaryotic virus display library, direct plasmid display library, etc.).

The target moieties can include any moiety that is expect to be bound or is capable of being bound by a protein. Such moieties include, but are not limited to proteins, nucleic acids, lipids, glycoproteins, carbohydrates, polysaccharides, and the like. The target moieties need not be limited to individual molecules. Thus, for example, it is possible to use cell surfaces, receptors, tissues, and the like as targets.

The target moieties are typically contacted with a library of potential binding partners (e.g. proteins that might be capable of binding to the target(s)). Such libraries typically comprise at least 100 different members, preferably at least 1000 different members, more preferably at least 10,000 and most preferably at least 10⁶, 10⁷, 10⁸, 10⁹ or 10¹⁰ different members. In certain embodiments, the libraries are cDNA libraries derived from a particular cell type/line, and/or a particular tissue, and/or a particular organism. The libraries, however, need not be limited to cDNA libraries. Other libraries include, but are not limited to antibody libraries (e.g. single chain antibody libraries), libraries of proteins randomized in one or more domains, libraries comprising shuffled polypeptides, and the like.

In preferred embodiments, the libraries of potential binding partners are provided on a “display vector”. Such display vectors include, but are not limited to phage-display vectors, bacterial display vectors (Fuchs et al. (1991) Biotechnology 9, 1369-1372), yeast display libraries (Boder and Wittrup (1997) Nat. Biotechnol. 15: 553-557), eukaryotic virus libraries (Kasahara et al. (1994) Science 266: 1373-1376), and direct plasmid display libraries (Cull et al. (1992) Proc. Natl. Acad. Sci. U.S.A. 89: 1865-1869), and the like. Suitable libraries also include in vitro display technologies (e.g. covalent display technology (CDT), polysome display, eukaryotic in vitro transcription/translation systems, RNA-peptide fusions, and the like (see, e.g., Fitzgerald (2000) Drug Discovery Today 5(6): 253-258, and references cited therein).

The ability to express polypeptides on the surface of bacteria or of viruses that infect bacteria (bacteriophage or phage) makes it possible to screen and one or more binding polypeptide or a libraries of greater than 10¹⁰ clones. To express polypeptides on the surface of phage (phage display), a nucleic acid encoding the polypeptide is inserted into the gene encoding a phage surface protein (e.g., pIII) and the polypeptide-surface fusion protein is displayed on the phage surface (McCafferty et al. (1990) Nature, 348: 552-554; Hoogenboom et al. (1991) Nucleic Acids Res. 19: 4133-4137). Since the polypeptides on the surface of the phage are functional, phage bearing binding polypeptides can be separated from non-binding phage by binding to a target (e.g. via antigen affinity chromatography) (see, e.g., McCafferty et al. (1990) Nature, 348: 552-554).

Phage display has been successfully applied to a wide range of peptides and proteins, including antibodies McCafferty et al. (1990) Nature, 348: 552-554), growth hormone (Bass et al. (1990) Proteins: Struct. Funct. Genet. 8(4): 309-314), DNA binding proteins (Jamieson et al. (1994) Biochem., 33(19): 5689-5695), enzymes (McCaffety et al. (1991) Protein Eng., 4(8): 955-961); Corey et al. (1993) Gene, 128(1): 129-134); Soumillion et al. (1994) J. Mol. biol., 237(4): 415-422), and macromolecular protease inhibitors (Roberts et al. (1992) Proc. Natl. Acad. Sci. USA, 89(6): 2429-2433); Pannekoek et al. (1993) Gene, 128(1): 135-140; Wang et al. (1995) J. Biol. Chem., 270(20): 12250-12256); Markland et al. (1996) Biochem., 35: 8058-8067; Markland et al. (1996) Biochem., 35: 8045-8057).

In certain embodiments, a phage display library utilizes so called “hyperphage”. In hyperphage, the number of single-chain antibody fragments (scFv) or other proteins, presented on filamentous phage particles can be increased by more than two orders of magnitude by using a newly developed helper phage (hyperphage). Hyperphage have a wild-type pIII phenotype and are therefore able to infect F+ Escherichia coli cells with high efficiency; however, their lack of a functional pIII gene means that the phagemid-encoded pIII-antibody fusion is the sole source of pIII in phage assembly. This results in a considerable increase in the fraction of phage particles carrying an the inserted protein on their surface (see, e.g., Rondot et al. (2001) Nature Biotechnology, 19(1): 75-78).

Similar to phage-display systems, methods are known to display heterologous proteins on the surface of bacteria. Thus, for example, U.S. Pat. No. 6,190,662 provides methods and vectors for obtaining surface expression of a desired protein or polypeptide in Gram-positive host organisms (e.g. a Lactococcus host). Similarly U.S. Pat. No. 5,348,867 teaches the expression of heterologous proteins on the surface of gram negative bacteria (e.g. E. coli, Pseudomonas aeruginosa, Haemophilus influenza, etc.).

Generally bacterial systems comprise tripartite chimeric genes. One segment of the tripartite gene is a targeting DNA sequence encoding a polypeptide capable of targeting and anchoring the fusion polypeptide to a host cell outer membrane. Targeting sequences are well known and have been identified in several of membrane proteins including Lpp. Generally, as in the case of Lpp, the protein domains serving as localization signals are relatively short. The Lpp targeting sequence includes the signal sequence and the first 9 amino acids of the mature protein. These amino acids are found at the amino terminus of Lpp. E. coli outer membrane lipoproteins from which targeting sequences may be derived include TraT, OsmB, NlpB and BlaZ. Lipoprotein 1 from Pseudomonas aeruginosa or the PA1 and PCN proteins from Haemophilus influenza as well as the 17 kDa lipoprotein from Rickettsia rickettsii and the H.8 protein from Neisseria gonorrhea and the like can be used.

A second component of the tripartite chimeric gene is a DNA segment encoding a membrane-transversing amino acid sequence. Transversing is intended to denote an amino acid sequence capable of transporting a heterologous or homologous polypeptide through the outer membrane. In preferred embodiments, the membrane transversing sequence will direct the fusion polypeptide to the external surface. As with targeting DNA segments, transmembrane segments are typically found in outer membrane proteins of all species of gram-negative bacteria. Transmembrane proteins, however, serve a different function from that of targeting sequences and generally include amino acids sequences longer than the polypeptide sequences effective in targeting proteins to the bacterial outer membrane. For example, amino acids 46-159 of the E. coli outer membrane protein OmpA effectively localize a fused polypeptide to the external surface of the outer membrane when also fused to a membrane targeting sequence. These surface exposed polypeptides are not limited to relatively short amino acid sequences as when they are incorporated into the loop regions of a complete transmembrane lipoprotein.

The third gene segment comprising the tripartite chimeric gene fusion is a DNA segment that encodes any one of a variety of desired heterologous polypeptides.

Other suitable display systems include, but are not limited to various ill vitro display technologies such as covalent display technology (CDT), polysome display, eukaryotic in vitro transcription/translation systems, RNA-peptide fusions, and the like (see, e.g., Fitzgerald (2000) Drug Discovery Today 5(6): 253-258, and references cited therein).

CDT exploits the properties of a replication initiator protein from the E. coli bacteriophage P2. The protein is the product of the viral Agene (P2A) and is an endonuclease that initiates a rolling circle replication process by binding to the viral origin (on) and introducing a single strand discontinuity (nick) in the DNA. The 3′-OH group that is exposed by the action of P2A is used to prime progeny DNA synthesis using the host replication machinery (Schnos and Inman (1971) J. Mol. Biol. 55: 31-38; Geisselsoder (1976) J. Mol. Biol. 100: 13-22; Chattoraj (1978) Proc. Natl. Acad. Sci., USA, 75:1685-1689). The nicking event also exposes a 5′ phosphate and this becomes covalently attached to a tyrosine residue in the active site of P2A (Lindahl (1970) Virology 42: 522-533; Liu et al. (1994) Nucleic Acids Res. 22: 5204-5210).

One further property of P2A that is exploited in the CDT system is that P2A exclusively attaches to the same molecule of DNA from which it has been expressed. The high fidelity of the cis activity and the fact that the recognition sequence for the covalent attachment, ori, occurs within P2A's own coding sequence (Schnos and Inman (1971) J. Mol. Biol. 55: 31-38; Geisselsoder (1976) J. Mol. Biol. 100: 13-22; Chattoraj (1978) Proc. Natl. Acad. Sci., USA, 75: 1685-1689; Lindahl (1970) Virology 42: 522-533; Liu et al. (1994) Nucleic Acids Res. 22: 5204-5210; Liu et al. (1993) J. Mol. Biol. 231: 361-374) enables pools of polypeptides that are genetically fused to P2A to be synthesized in vitro such that they also become covalently attached to their own coding sequences.

To operate CDT, a pool of DNA molecules is prepared, each containing the coding sequence of P2A fused to the coding sequence for one of a diverse population of potential binding moieties (linear peptides or protein domains). The DNA pool is transcribed and translated concurrently in vitro using an E. coli S30 lysate and, because of the cisactivity of P2A, each DNA molecule becomes covalently tagged with its own expressed gene product. The protein-DNA complexes are then subjected to various screening/selection strategies.

Polysome display systems work by transcribing and translating DNA templates in vitro under conditions that enable the isolation of stable mRNA-ribosome-nascent polypeptide complexes (Schaffitzel et al. (1999) J. Immunol. Methods 231: 119-135). This is achieved by controlling the concentration of magnesium ions (to stabilize the ribosome particle) and by either terminating polypeptide elongation by the addition of chloramphenicol or cooling down the translation products of mRNA templates that lack stop codons. Target-specific polysome complexes are retained on an appropriately derivatized solid surface and the co-selected mRNAs released by dissociation of ribosomes using ethylene diamine tetraacetate (EDTA). These are then recovered by reverse transcription (RT) and PCR for further manipulation.

Another in vitro display system uses a puromycin molecule to provide a covalent linkage between mRNA molecules and their encoded polypeptides (Roberts and Szostak (1997) Proc. Natl. Acad. Sci., USA, 94: 12297-12302). Puromycin is an antibiotic that mimics the aminoacyl end of tRNA and functions by entering the ribosomal A-site and forming an amide linkage with nascent polypeptide through the peptidyl transferase activity of the ribosome.

In the RNA-peptide fusion system, the puromycin is attached to the 3′ end of a single-stranded DNA linker that is in turn ligated to the 3′ end of the library-encoding mRNA. When the mRNA is translated in vitro, a ribosome reaches the junction between the mRNA and the DNA linker and stalls. The puromycin can then enter the ribosomal A-site and form a stable amide linkage with the encoded peptide. A library pool of mRNA-DNA-puromycin molecules can therefore be translated in vitro and purified RNA-peptide complexes incubated with a target molecule for screening. As with the polysome display system, retained complexes are recovered for further manipulation by RT-PCR.

These embodiments of display libraries are illustrative and not intended to be limiting. Other suitable display library formats will be known to those of skill in the art.

In a particularly preferred embodiment, display libraries are created that express a library of cDNAs, or other potential binding proteins as described herein. Nucleic acids cDNAs encoding all the desired potential binding proteins can be prepared and inserted into the “vehicle(s) comprising the display library.

The inserted nucleic acids are made according to methods well known to those of skill in the art. For example, in one approach, the nucleic acids can be chemically synthesized using nucleotide reagents. However, in a particularly preferred embodiment, however, the nucleic acids are created using standard cloning techniques, e.g., amplification (e.g., PCR) cloning with appropriate primers. Detailed protocols for the production of libraries using phage display technology are provided in Example 1.

Selecting Bound Members of the Phage- or Bacterial-Display Library.

In preferred methods, members of the display library that bind to said one or more target proteins are selected to provide a preselected set of potential binding proteins. Methods of selecting bound phage-display or bacterial display members or other display library members are well known to those of skill in the art.

In a particularly preferred embodiment the target moiety (e.g. protein, DNA, etc.) is provided attached to a solid support/substrate. In such instances, after the phage- or bacterial-display library is contacted with the target(s), the unbound phage can be washed away and/or the substrate bearing the target(s) bound by phage can be separated from the solution containing the library. Repetitive wash steps will eliminate unbound library members.

Suitable supports for the attachment of target moieties include, but are not limited to the surfaces of wells, capillaries, planar surfaces, particulate materials (beads, etc), slurries, gels, and the like. Preferred materials include, but are not limited to magnetic beads, glass, plastic, ceramics, metals, various resins, membranes, and the like. The target moiety is coupled to the surface according to standard methods well known to those of skill in the art.

The target moieties can be directly coupled to the substrate or can be joined to the substrate through a linker. The procedure for attaching a target moiety to the substrate will vary according to the chemical structure of the moiety. Proteins contain a variety of functional groups (e.g., —OH, —COOH, —SH, or —NH₂) groups, that are available for reaction with a suitable functional group on a surface or a linker to bind the target thereto. Alternatively, the target moiety can be derivatized to expose or attach additional reactive functional groups. The derivatization may involve attachment of any of a number of linker molecules such as those available from Pierce Chemical Company, Rockford Ill. A bifunctional linker having one functional group reactive with a group on a particular target moiety and another group reactive with a group on the substrate can be used to anchor the target moiety.

In certain embodiments, the target moieties can be attached to the surface by simple adsorption.

In other embodiments, the target moieties can be provided in solution and contacted to the members of the phage- or bacterial display library also in solution. In such instances, the target moiety can comprise a domain (tag) that can be specifically captured/bound by an affinity reagent (e.g. an antibody, ligand, etc.). Alternatively, the target moiety can be attached to a tag (e.g. an affinity tag) that can be captured by an affinity reagent.

Affinity tags are well known to those of skill in the art. Such tags include, but are not limited to biotin with avidin/streptavidin, ligands and their cognate receptors, particularly haptens and antibodies, polyhistidine with Ni-NTA, glutathione S-transferase (GST) and glutathione, epitopes and cognate antibodies, and the like.

Certain affinity tags include epitope tags. Epitope tags are well known to those of skill in the art. Moreover, antibodies (intact and single chain) specific to a wide variety of epitope tags are commercially available. These include but are not limited to antibodies against the DYKDDDDK (SEQ ID NO:5) epitope, c-myc antibodies (available from Sigma, St. Louis), the HNK-1 carbohydrate epitope, the HA epitope, the HSV epitope, the His₄, His₅, and His₆ epitopes that are recognized by the His epitope specific antibodies (see, e.g., Qiagen), and the like.

In certain preferred embodiments, the target moiety is tagged with a hexahistidine (His₆) epitope tag that is bound by a Cu, Ni, or Co complex. One particularly preferred complex for binding His₆ tags is Ni-NTA (Ni-nitrilotriacetic acid). In certain particularly preferred embodiments, the affinity tag is a biotin which can then be captured by avidin, streptavidin, or variants thereof.

The affinity tagged target moiety is contacted with the phage- or bacterial display library, e.g., in solution. Where suitable binding polypeptides exist in the library the target moieties are bound thereby forming a target moiety/binding polypeptide complex. The bound complexes can be recovered from solution phase by the use of an affinity matrix (e.g. a resin or other substrate attached to a ligand that binds to the affinity tag on the target moieties). Once isolated, the assay proceeds as with the target moieties provided attached to a substrate.

The target moieties binding polypeptides are isolated thereby providing a preselected set of potential binding proteins. The bound library members can then be separated (e.g. eluted) from the target moieties by the use of standard methods well known to those of skill in the art (e.g. using denaturing reagents, high salt, chaotropic reagents, and the like).

Contacting Members of the Preselected Set of Potential Binding Partners with one or more Target Proteins.

In preferred embodiments, the methods of this invention involve a second screening assay. In this assay, the preselected set of potential binding partners is again probed with the one or more target moieties to identify which members of the potential binding partners bind (e.g. specifically bind) to particular target moieties.

In preferred embodiments, the second assay is a different format from the first assay. In particularly preferred embodiment, however, the preselected members of the display library (preselected set of potential binding partners) is provided in a “spatially addressable” format. This permits individual members of the library that screen positive (for specific target binding) in the second screen to be detected and discriminated from each other. Such assays are thus preferably “inclusive” selecting for all binding partners rather than “exclusive” screening for a single one or few optimal binding partners.

Numerous assays are suitable. In one particular preferred embodiment, the second screen is a conventional cDNA expression library screening method. In this instance, the expressed cDNA library is immobilized on a solid substrate (e.g. blotted onto a membrane) and then probed with the one or more targets. Targets that specifically bind to the library members are identified and the binding members are optionally sequenced.

In preferred embodiments, the target moieties are labeled with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P) enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure.

The label can be coupled to the target moiety prior to, during, or after the binding assay. So called “direct labels” are detectable labels that are directly attached to or incorporated into the target moiety prior to the binding assay. In contrast, so called “indirect labels” are joined to the target moiety/binding protein complex after binding. Often, the indirect label is attached to a second binding moiety that specifically binds to the target moiety or to a tag attached thereto. Thus, for example, the target moiety can be biotinylated before the screening assay. After hybridization, an avidin-conjugated fluorophore will bind the biotin bearing complexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

It will be recognized that fluorescent labels are not to be limited to single species of organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

Kits.

In still another embodiment, this invention provides kits for the practice of the methods described herein. Preferred kits include one or more components of a display library (e.g. phage display, bacterial display, yeast display, eukaryotic virus display library, direct plasmid display library, etc.) and instructional materials providing protocols for the assays disclosed herein.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

TAIS Database.

In certain embodiments, this invention contemplates the use of a database to permit storage, retrieval, and management of TAIS data. Thus, for example, such a database can records showing amino acid sequence or identity of a member of a set of potential binding partners or proteins that interact with a one or more particular targets.

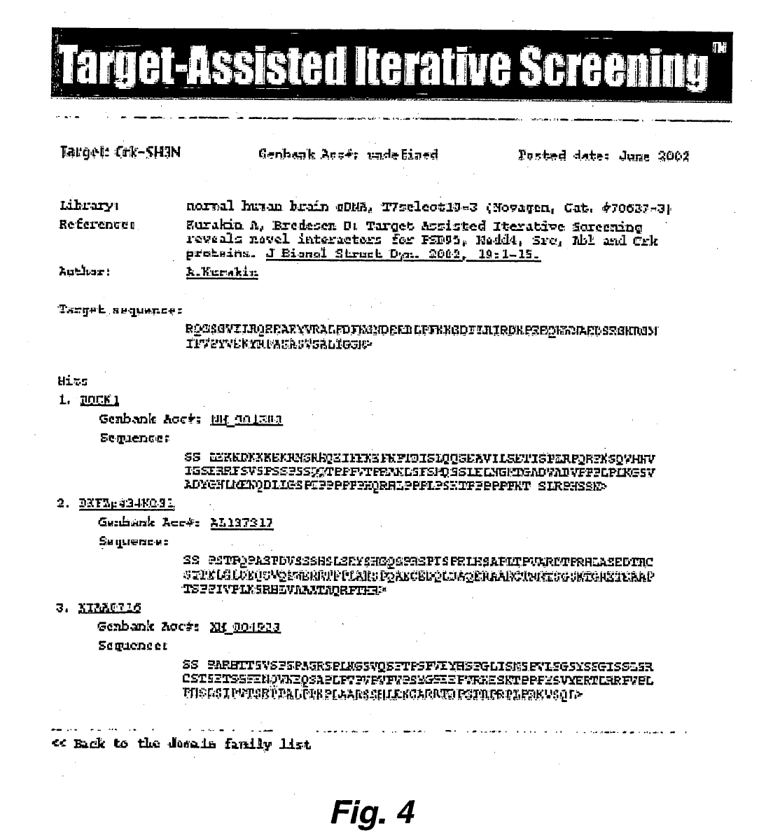

An illustration of an entry in such a database is provided in FIG. 4. The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### EXAMPLES

The following examples are offered to illustrate, but not to limit the claimed invention.

### Example 1

### Use Of TAIS To Study Protein-Protein Interactions

Results from screening of a T7 cDNA library derived from the normal human brain (NOVAGEN. Cat. #70637-3. (2001)) are presented and discussed below to demonstrate the potential of TAIS in mapping of protein-protein interactions. SH3, PDZ and WW domains of the Abl, Src, Crk, PSD95 and Nedd4 proteins have been used as test targets. In total, 12 novel putative and 2 previously described interactions have been identified by TAIS for these well studied protein interaction modules.

Combinatorial peptide libraries displayed on the phage or synthesized chemically have proved to be an excellent tool to define ligand preferences of peptide interaction modules (Cheadle et al. (1994) J Biol Chem 269: 24034-24039; Rickles et al. (1994) Embo J 13: 5598-5604; Sparks et al. (1996) Proc. Natl. Acad. Sci., USA, 93: 1540-1544; Kay et al. (2000) FEBS Lett 480, 55-62). The recognition consensus of an individual domain can be inferred by analyzing amino acid sequences of peptides selected from a random peptide library by the domain in question (Sparks et al. (1996) Proc. Natl. Acad. Sci., USA, 93: 1540-1544; Kay et al. (2000) FEBS Lett 480, 55-62). Defining the recognition consensus facilitates identification of potential interacting partners of the domain in protein databases (Kurakin et al. (1998) J Pept Res 52: 331-337) and/or mapping of its interaction sites within known partners (Id.). However, since combinatorial peptide repertoires are artificial, it is not clear how accurate the inferred consensus reflects natural interacting sequences and, often, the consensus defined in this way is too broad to limit the number of potential interactors in databases to a manageable quantity. The advent of cDNA libraries displayed on phage provides an opportunity to search natural peptide repertoires in order to map interacting partners and to refine recognition consensuses.

We demonstrate here that TAIS when applied to cDNA libraries allows rapid and simultaneous exploration of combinatorial and natural peptide repertoires with protein interaction modules as targets. This feature makes TAIS an efficient tool for both direct mapping of protein-protein interactions and studies aiming to characterize molecular recognition properties of protein interaction modules.

Results and Discussion

The Method.

A cDNA library derived from normal human brain was used in all presented screens (NOVAGEN. Cat. #70637-3. (2001), Novagen, Inc.). The library was generated using purified poly(A)⁺ mRNA from the brain tissue as a template to create first strand cDNAs, which in turn served as templates for the synthesis of double stranded cDNA fragments. In both cases priming was random, thus the size and composition of resultant cDNA inserts vary greatly. The cDNA fragments longer than 300 base pair were directionally ligated to the C-terminus of gene product 10 of the lytic bacteriophage T7. Therefore, upon phage assembly a fragmented tissue-specific proteome is displayed on the surface of T7 phage as a C-terminal fusion to the major phage coat protein (NOVAGEN. OrientExpress cDNA Manual, TB247. (1999)). The reported diversities of tissue specific cDNA libraries from this source are in the order of 5×10⁷ primary recombinants, suggesting that even rare mRNA sequences are represented in these libraries with high probability (Soares et al. (1994) Proc. Natl. Acad. Sci., USA, 91: 9228-9232; Maniatis, et al. (1982) Molecular cloning. A Laboratory Manual. p. 225. (Cold Spring Harbor)). An important point to keep in mind is that theoretically, due to random priming, only one-third of all cDNA inserts result in the display of peptide sequences from the proteome. Two-thirds can be considered as “random” peptides originating from frameshifts upon ligation. In reality, the proportion of proteome sequences in the library is even less, due to priming from untranslated regions of mRNA. This structure of the library, however, is of great advantage when it is used to characterize ligand preferences of peptide interaction domains, for it allows parallel exploration of natural and artificial peptide repertoires.

To evaluate the new screening method, representatives of three families of peptide interaction modules, PDZ, SH3 and WW, were chosen as test targets. The domains were derived from well-known proteins, such as PSD95, Src, Abl, Crk and Nedd4, for the following reasons: all five proteins have been the subjects of extensive protein interaction studies for a number of years performed by different groups and by different methods. In fact, PDZ and SH3 domains were first described in PSD95 and Src proteins, respectively, about a decade ago (Cho et al. (1992) Neuron 9: 929-942; Koch et al. (1991) Science 252: 668-674). A number of protein interactions mediated by these domains have been reported in the literature (Barfod et al. (1993) J Biol Chem 268: 26059-26062; Weng et al. (1994) Mol Cell Biol 14: 45094521; Kapeller et al. (1994) J Biol Chem 269: 1927-1933; Gout et al. (1993) Cell 75: 25-36; Weng et al. (1993) J Biol Chem 268: 14956-14963; Ren et al. (1993) Science 259: 1157-1161; Gertler et al. (1995) Genes Dev 9: 521-533; Ren et al. (1994) Genes Dev 8: 783-795; Knudsen et al. (1994) J Biol Chem 269: 32781-32787; Hasegawa et al. (1996) Mol Cell Biol 16: 1770-1776). In addition, ligand preferences of the tested domains have been characterized by screening of artificial peptide repertoires (Cheadle et al. (1994) J Biol Chem 269: 24034-24039; Rickles et al. (1994) Embo J 13: 5598-5604; Sparks et al. (1996) Proc. Natl. Acad. Sci., USA, 93: 1540-1544 Sparks et al. (1994) J Biol Chem 269: 23853-23856; Rickles et al. (1995) Proc. Natl. Acad. Sci., USA, 92: 10909-10913; Yu et al. (1994) Cell 76: 933-945; Feng et al. (1994) Science 266: 1241-1247; Musacchio et al. (1994) Nat Struct Biol 1: 546-551; Wu et al. (1995) Structure 3: 215-226). The reported interactions were meant to serve as a positive control while known recognition consensuses of tested domains were expected to match sequences in peptides selected by TAIS from a cDNA library.

PDZ Domains of PSD95.

PDZ domains were originally described as 80-100 amino acid conserved repeats within the post-synaptic density 95 protein (PSD95) (Cho et al. (1992) Neuron 9: 929-942; Kornau et al. (1997) Curr Opin Neurobiol 7: 368-373). The prototypical PDZ domain protein PSD95 comprises three PDZ domains at the N-terminus followed by an SH3 domain and an inactive guanylate kinase domain (Cho et al. (1992) Neuron 9: 929-942). By providing an architectural and functional scaffold via its multiple protein interaction modules, it is thought to orchestrate assembly and function of molecular complexes responsible for neurotransmission and synaptic plasticity at the post-synaptic membranes (Kennedy (2000) Science 290: 750-754; El-Husseini et al. (2000) Science 290: 1364-1368).

In their classical mode, PDZ domains recognize and bind to the extreme C-terminal sequences of interacting partners with reported affinities from high nanomole to low micromole range (Niethammer et al. (1998) Neuron 20: 693-707; Songyang et al. (1997) Science 275: 73-77). Specificity of binding within the PDZ family is thought to be defined by 3-5 amino acids preceding the C-terminal residue (Songyang et al. (1997) Science 275: 73-77; Stricker et al. (1997) Nat Biotechnol 15: 336-342; Doyle et al. (1996) Cell 85: 1067-1076) Ligand preferences of different PDZ domains have been studied mostly with chemically synthesized, rather than displayed peptide libraries, due to historical difficulties in displaying free carboxy-termini on the filamentous phage (Songyang et al. (1997) Science 275: 73-77; Stricker et al. (1997) Nat Biotechnol 15: 336-342; Doyle et al. (1996) Cell 85: 1067-1076; Hoffmuller et al. (1999) Angew. Chem. Int. Ed. 38: 2000-2004). Analysis of the ligand preferences of several PDZ domains resulted in inferred recognition consensus sequences, which, though fitted well when compared to natural binding sites discovered by other methods, were of limited predictive power due to a too broadly defined consensus.

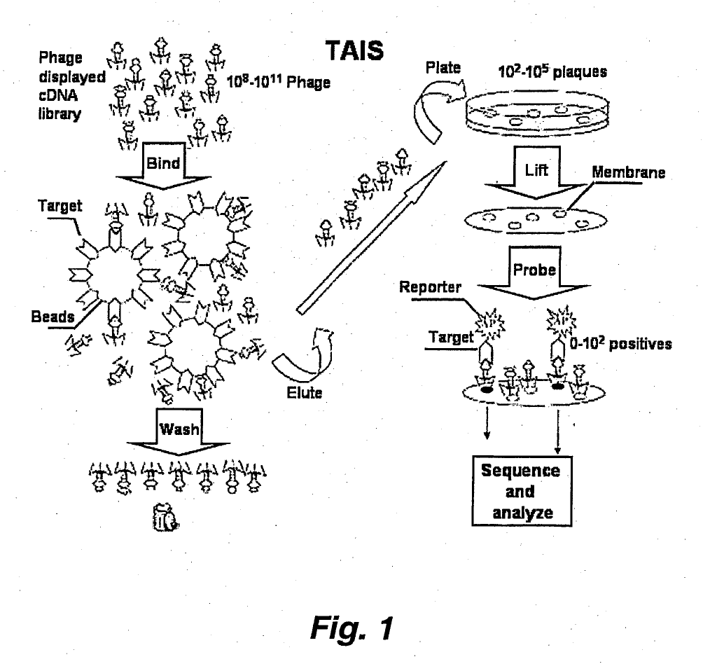

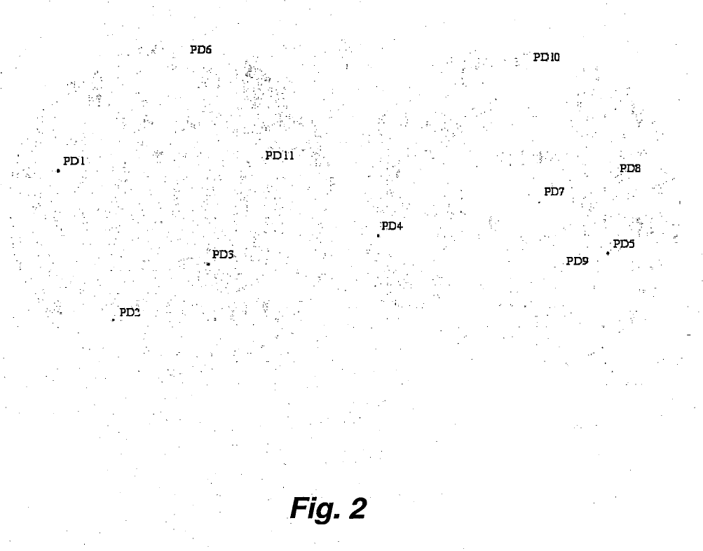

A cDNA human brain library displayed on the T7 phage was TAISed with the N-terminal fragment of PSD95 comprising three PDZ domains as a target (PSD95-PDZ(1+2+3)). The pre-selected cDNA library formed about 1500 plaques on a bacterial lawn, when plated on two 150 mm Petri dishes. 11 clones gave positive signals on the membranes after plaque lift and screening of membranes with biotinylated PSD95-PDZ(1+2) complexed to streptavidin-alkaline phophatase (AP) conjugate (see FIG. 2).

Sequences of the peptides displayed on the phages that gave positive plaques are numbered PD1 through PD11 and shown in Table 1, together with their relative affinity ranks towards PSD95-PDZ(1+2+3).

TABLE 1

Results of screening of a phage-displayed human brain
cDNA library with an N-terminal fragment of PSD95
comprising its three PDZ domains. Sequences of
polypeptides displayed by phages from positive
plaques along with their relative affinity ranks
towards the target and identities of the respective
cDNA inserts. FS - frameshift, ? - undefined, DGKζ -
diacylglycerol kinase zeta, UTR - untranslated region,
“>” - denotes free carboxylate group.

| | | | | |
|---|---|---|---|---|
| | | | | SEQ |
| Phage Clone | Displayed Peptide | Binding | cDNA | ID NO |

| | | | | |
|---|---|---|---|---|
| PD1, PD4, | SRSTWATWQSPIYTKKPKTSQV> | ++++++++ | ? | 6 |
| PD5 | | | | |
| PD2 | SKIKYFRESII> | ++++++++ | ? | 7 |
| PD3 | SSRQHYQMIQREDQETAV> | ++++++++ | DGK ζ | 8 |
| PD6 | SSLRLETGV> | + | ? | 9 |
| PD7 | LRNGRRECHIHLWKQRGQMRISAV> | +++ | ? | 10 |
| PD8, PD9 | PASAQPAAGDPVPAPAVLLGWTLV> | ++ | FS | 11 |
| PD10 | SSRKCRQCFHKSKCTVI> | + | UTR | 12 |
| PD11 | SSLV> | +/− | FS | 13 |
| Minimum | xRxSxV> | | | 14 |
| Consensus | K T I | | | |
| Refined | KxxRESxV> | | | 15 |
| Consensus | R K T I | | | |
| (PD1-PD5) | | | | |

The minimum consensus sequence of peptides that bound PSD95-PDZ(1+2) can be readily defined as (R/K)-x-(S/T)-x-(V/I)-COOH (SEQ ID NO:16). This consensus matches well with C-terminal sequences of known interacting partners of PSD95, such as inward rectifier K⁺ channel (Kir2.3: NISYRRESAI-COOH, SEQ ID NO:17) (Cohen et al. (1996) Neuron 17: 759-767), embryonic skeletal muscle sodium channel (SkM2: SPDRDRESIV-COOH, SEQ ID NO:18) (Gee et al. (1998) J Neurosci 18:128-137) and Shaker-type potassium channel (Kv1.4: SNAKAVETDV-COOH, SEQ ID NO:19) (Kim et al. (1995) Nature 378: 85-88). It is also notably similar to the consensus previously reported for syntrophin PDZ domains, (R/K)-E-(S/T)-x-V-COOH (SEQ ID NO:20, Gee et al. (1998) J Neurosci 18: 128-137) (see below). Significantly, 2 out of the 3 strongest binders have a conserved glutamate at ligand position −3 and all of the strongest binders (PD1, PD2, PD3) have a positively charged residue at the position −7*, lysine or arginine. (Conventionally, residues of a peptide ligand for PDZ domains are numbered so that the extreme C-terminal residue position is designated as 0 and positions of preceding residues towards the N-terminus are −1, −2, −3, 4 and so on). Therefore, a refined binding consensus of PSD95-PDZ(1+2) can be described as (K/R)-x-x-(R/K)-E-(S/T)-x-(V/I)-COOH (SEQ ID NO:21). It should be noted that residues of PDZ ligands distant from the C-terminus, such as −7 or −8 positions, have been implicated previously as contributing to the binding specificity, at least in the cases of some PDZ domains (Niethammer et al. (1998) Neuron 20: 693-707; Songyang et al. (1997) Science 275: 73-77). Collectively, our data and that of others suggest that the recognition mechanism of PDZ domains may be more complex than currently believed, and may involve additional specificity determinants proximal to the C-terminal five amino acids.

The cDNA library can be viewed as a combinatorial library that is highly enriched in natural peptide sequences. The latter provide a unique internal reference about physiologically relevant affinities and specificities when the library is assayed for the interaction with a target protein. Taking into account these considerations, we believe that PD1 and PD2 peptides, that bound strongly to PSD95-PDZ(1+2+3), may represent novel proteins that interact with PSD95. The nucleotide sequences of PD1 and PD2 inserts match a number of human ESTs and genomic sequences with no assigned open reading frame (not shown). The biochemical characterization of corresponding full-length cDNA products can substantiate this putative activity/function.

When used in pattern searches of the SWISS-PROT database, (K/R)-x-x-(R/K)-E-(S/T)-x-(V/I)-COOH (SEQ ID NO:22) consensus matches sequences in about 30 proteins, a reasonable number to assess experimentally. Therefore, the potential interacting partners that are missed in a physical screen due to their absence, low abundance or sensitivity to proteolysis can be retrieved by bioinformatic tools using the recognition consensus of the target refined by TAIS.

We have used the PSD95-PDZ(1+2+3) recognition consensus defined by TAIS, [KR]-x-x-[QRK]-E-[ST]-x-[VI]-COOH (SEQ ID NO:23), in homology searches of SWISS and TrEMBL databases. Proteins with the C-termini conforming to the query consensus are grouped below according to their functionality or their host (see, e.g. Table 2 and Table 3).

TABLE 2
Proteins with the C-termini conforming to the query consensus (TAIS,
[KR]—x—x-[QRK]-E-[ST]-x—[VI]—COOH, SEQ ID
NO: 24) grouped according to their functionality or their host.

| | | SEQ |
|---|---|---|
| | | ID |
| Protein | SEQUENCE | NO |
| Receptors: | RNLRETDI | 25 |
| A1AD Rabbit 002666 | rabbit | |
| Alpha-1D adrenergic receptor) | LADYSNLRETDI | 26 |
| Oryctolagus cuniculus (Rabbit) | human | |
| 569-576 | | |
| Microtubule Associated Motor | | |
| KF1B HUMAN O60333 | KAGRETTV | 27 |
| Kinesin-like protein KIF1B (Klp) | | |
| Homo sapiens (Human) | | |
| SPLICE ISOFORM 3 1146-1153 | | |
| O60575 (KF1B MOUSE) | KAGRETTV | 28 |
| Kinesin-like protein KIF1B | | |
| [Mus musculus (Mouse)] | | |
| SPLICE ISOFORM 3 OF Q60575 | | |
| 1143-1150 | | |
| O9H8Z3 CDNA FLJ13122 fis, | KAGRETTV | 29 |
| clone NT2RP3002688 weakly similar | | |
| to mouse kinesin-like protein | | |
| (Kif1b) [Homo sapiens (Human)]. | | |
| 122-129 KAGRETTV | | |
| AAK33008 | KGSRETAV | 30 |
| Kinesin-like protein Kif1b alpha | | |
| [Brachydanio rerio (Zebrafish) | | |
| (Danio rerio)]. 1154-1161 | | |
| KGSRETAV | | |
| HUMAN VIRAL PROTEINS. | | |
| TAT HTL1A P03409 | KHFRETEV | 31 |
| Trans-activating transcriptional | | |
| regulatory protein (X-LOR | | |
| protein) (PX protein). Human | | |
| T-cell leukemia virus type I | | |
| (strain ATK & Caribbean isolate) | | |
| (HTLV-I) 351-358 | | |
| VE6 HPV45 P21735 | RRRRETQV | 32 |
| E6 protein. Human papillomavirus | | |
| type 45 (conforms for types 56, | | |
| 68, 70, ME180, 151-158 | | |
| O73280 | KRPRESDI | 33 |
| GAG polyprotein [Contains: core | | |
| protein(s) P24] (Fragment). | | |
| Human immunodeficiency virus | | |
| type 2118-125 | | |
| US32 HCMVA P09708 | RRHRETYV | 34 |
| Hypothetical protein HHRF7. | | |
| Human cytomegalovirus (strain | | |
| AD169) 176-183 | | |
| HUMAN BACTERIAL | | |
| PARASITES'S PROTEINS | | |
| Y3C2_MYCTU O53600 | RGERESFV | 35 |
| Hypothetical 13.3 kDa protein | | |
| Rv3922c. [Mycobacterium | | |
| tuberculosis] 113-120 | | |
| O9R886 | RQNKETKI | 36 |
| Hypothetical 3.6 kDa protein | | |
| (Fragment). Chlamydia | | |
| trachomatis 23-30 | | |
| O84715 | KKRKESLV | 37 |
| ROD SHAPE PROTEIN-SUGAR | | |
| KINASE. Chlamydia trachomatis | | |
| 359-366 | | |
| O9PLL7 | KKRKESLV | 38 |
| Cell shape-determining protein | | |
| MreB. Chlamydia muridarum | | |
| 359-366 | | |
| SIGNALING (EXOCYTOSIS - | | |
| RAL FAMILY BINDING PROTEIN) | | |
| O62796 | KDRKETPI | 39 |
| RalBP1 Rattus norvegicus (Rat) | | |
| 640-647 | | |
| O15311 | RDRKETSI | 40 |
| RLIP76 protein (Similar to ra1A | | |
| binding protein 1). | | |
| Homo sapiens (Human) 648-655 | | |
| O62172 | KDRKETPI | 41 |
| RIP1 protein. | | |
| Mus musculus (Mouse) 641-648 | | |
| O9DDA3 | KDWKETLI | 42 |
| RalB-binding protein (Fragment). | | |
| Xenopus laevis (African clawed | | |
| frog) 604-611 | | |
| SIGNALING (SECOND | | |
| MESSAGER METABOLISM) | | |
| O13574 (KDGZ HUMAN) | REDQETAV | 43 |
| Diacylglycerol kinase, zeta | | |
| (EC 2.7.1.107) Diglyceride kinase) | | |
| (DGK-zeta) (DAG kinase zeta) | | |
| [Homo sapiens (Human)]. | | |
| 1110-1117 | | |
| SPLICE SHORT ISOFORM | REDQETAV | 44 |
| OF O13574 921-928 | | |
| O08560 (KDGZ RAT) | REDQETAV | 41 |
| Diacylglycerol kinase, zeta | | |
| (EC 2.7.1.107) (Diglyceride kinase) | | |
| (DGK-zeta) (DAG kinase zeta) | | |
| (DGK-IV) (104 kDa diacylglycerol | | |
| kinase) [Rattus norvegicus | | |
| (Rat)]. 922-929 | | |
| O91YS0 | REDQETAV | 45 |
| Similar to diacylglycerol kinase | | |
| (Fragment) [Mus musculus | | |
| (Mouse)]. 451-458 | | |

TABLE 3
Other proteins with the C-termini conforming to the
query consensus (TAIS, [KR]—x—x-[QRK]-E-[ST]-x—[VI]—COOH,
SEQ ID NO: 46).

| Accession No | Description |
|---|---|
| Q920A7 | (AF31_MOUSE) AFG3-like protein 1 |
| | (EC 3.4.24.-) [Mus musculus (Mouse)]. |
| P51464 | (ARLY_RANCA) Argininosuccinate lyase |
| | (EC 4.3.2.1) (Arginosuccinase) (ASAL) |
| | [Rana catesbeiana (Bull frog)]. |
| Q9P280 | KIAA1448 protein (Fragment) |
| | [Homo sapiens (Human)]. |
| Q9UIZ9 | Cellular DNA/human papillomavirus |
| | proviral DNA [Homo sapiens (Human)]. |
| Q9VHT6 | CG9626 protein [Drosophila melanogaster |
| | (Fruit fly)]. |
| Q9TR85 | DNA ligase II (Fragment) [Bos taurus |
| | (Bovine)]. |
| Q9LVM3 | Genomic DNA, chromosome 5, P1 clone: |
| | MCK7 [Arabidopsis thaliana (Mouse-ear |
| | cress)]. |
| Q90YA3 | 6-phosphofructokinase [Gallus gallus |
| | (Chicken)]. |
| AAM32072 | Conserved protein [Methanosarcina mazei |
| | Goe1]. |
| YC11_AQUAE | Hypothetical protein AQ_1211. [Aquifex |
| O67264 | aeolicus] |
| O29148 | DNA-DIRECTED RNA POLYMERASE, |
| | SUBUNIT E′ (RPOE1) |
| | [Archaeoglobus fulgidus]. |
| Q08300 | CHROMOSOME XV READING FRAME ORF |
| | YOL159C. [Saccharomyces cerevisiae] |
| | (Baker's yeast) |
| O80591T27I1.2 | protein. [Arabidopsis thaliana] |
| | (Mouse-ear cress) |

The interspecies conservation of the TAIS-defined PSD95-PDZ(1+2+3) recognition consensus at the C-termini of diacylglycerol kinase zeta (DGKζ), kinesin-like protein KIF1B and Ral-binding protein makes them strong candidates for being physiological interacting partners of PSD95. Notice that the C-terminus of human DGKζ interacted in vitro with PSD95-PDZ(1+2+3). The presence of PSD95-binding sequences at the C-termini of proteins from different Chlamydia strains may indicate on interesting and unexpected molecular connections exploited by this intracellular parasite, which is implicated in a host of human ailments such as trachoma, arthritis, Alzheimer's disease among others.

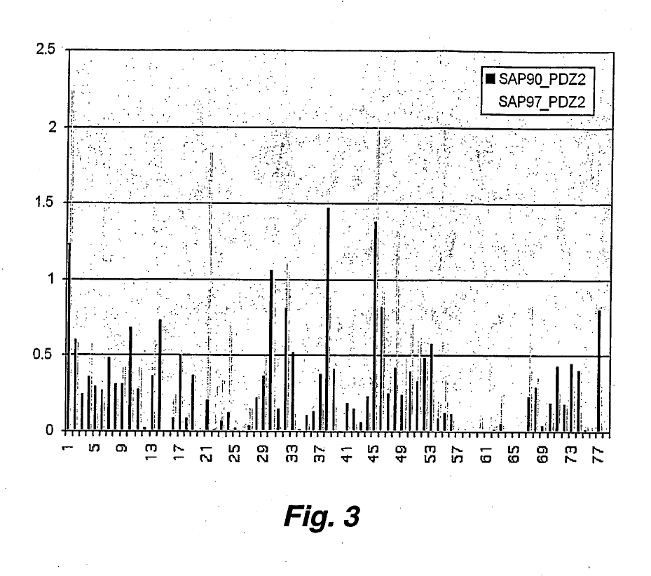

FIG. 3 illustrates another example of PDZ domain profiling. The x-axis shows an array of individual phages selected to bind a number of different PDZ domains, while the y-axis shows the relative affinities of individual phages to the 2nd PDZ domains from SAP97 and SAP90 in an ELISA-type assay. Table 4 illustrates PDZ2 domain best binders.

TABLE 4

SAP97_PDZ2 domain best binders and
SAP90_PDZ2 domain best binders.

SEQ ID
NO

SAP97_PDZ2 domain best binders

| | | |
|---|---|---|
| #1 | PGQHGESPSLLKTHKKISWV> | 47 |
| #45 | EKCHQSYSHSIYERKKWTDV> | 48 |
| #21 | SQPQEPVPVALQGVRRETRV> | 49 |
| #48 | GLGKSSRSLWGGEWHLETYV> | 50 |
| #32 | WAGPRKAGPLGAAPGRATLV> | 51 |
| #30 | NCCVNEPDTLLNLSPRWTMV> | 52 |
| consensus | WTxV | 53 |
| | E | |
| | I | |
| | A | |

SAP90_PDZ2 domain best binders

| | | |
|---|---|---|
| #38 | PARPTWGNSISTKNTKISWV> | 54 |
| #45 | EKCHQSYSHSIYERKKWTDV> | 55 |
| #1 | PGQHGESPSLLKTHKKISWV> | 56 |
| #30 | NCCVNEPDTLLNLSPRWTMV> | 57 |
| #32 | WAGPRKAGPLGAAPGRATLV> | 58 |
| #46 | RVPRRGQDFCSGFPGCWTQV> | 59 |
| consensus | WTxV> | 60 |
| | IS | |
| | A | |

Peptides that bound strongly to SAP97_PDZ2,
but only weakly to SAP90_PDZ2 share glutamic
acid (E) at position “−3” (shown in bold)

| | | |
|---|---|---|
| #21 | VSQPQEPVPVALQGVRRETRV> | 61 |
| #67 | ARAGGGFEDASLGFGGRETAV> | 62 |
| #48 | GLGKSSRSLWGGEWHLETYV> | 63 |

>indicates carboxy terminus.

Thus, despite the high degree of similarity between PDZ2 domains of SAP90 and SAP97 (84% of identity and 92% of similarity) their binding specificities are overlapping, but not identical.

The accumulation and arraying of peptides (on phages) that have been preselected to bind PDZ domains allows the rapid cross-comparison of PDZ domain specificities to reveal their unique binding characteristics. A rrays of PDZ-binding phages are easily propagated in multi-well formats and can be used for the rapid characterization of novel PDZ domains omitting library screening.

DGKζ.

Diacylglycerol kinase zeta (DGK ζ) was identified in the screen as a novel putative interacting partner of PSD95. DGKs metabolize a lipid second messenger diacylglycerol (DAG), thus negatively regulating DAG-induced cell responses (Topham et aaL (1999) J Biol Chem 274: 11447-11450; Sanjuan (2001) J Cell Biol 153:207-220). DAG is generated by phosphoinositide-specific phospholipase C (PLC) isoforms and accumulates locally and transiently upon activation of a large number of growth factor and other cell surface receptors (Bishop and Bell (1986) J Biol Chem 261: 12513-12519; Rhee (2001) Annu Rev Biochem 70: 281-312). We speculate that PSD95 by interacting with the C-terminus of DGK ζ maintains a diacylglycerol kinase activity as a component of signal-processing machinery at the postsynaptic membranes of glutamatergic synapses, where group I metabotropic glutamate receptors (mGluRs) (Skeberdis et al. (2001) Neuropharmacology 40: 856-865; Hannan et al. (2001) Nat Neurosci 4: 282-28.8; Reyes-Harde et al. (1998) Neurosci Lett 252: 155-158) and, conceivably, tyrosine kinases such as ErbB4 (Huang et al. (2000) Neuron 26: 443-455; Huang et al. (2001) J Biol Chem 276: 19318-19326) are coupled to the PLC cascade. Localization of DGK in close proximity to its substrate, rather than its shuttling between the cytosol and membrane, would allow higher frequencies of signal relay dependent on DAG generation.

Interestingly, DGK ζ has been recently reported by Gee and colleagues to bind via its C-terminus to PDZ domains of syntrophins (Hogan et alo. (2001) J Biol Chem 276: 26526-26533). Based on the similarities in critical residues between syntrophin PDZ domains and the second PDZ domain of PSD95, as well as their cross-reactivity to a number of targets, the same authors earlier suggested that these domains may compete for similar ligands (Gee et al. (1998) J Biol Chem 273: 21980-21987). Their suggestion is compatible with our findings as well as with the recently reported solution structure of the PSD95-PDZ2 domain, which most closely resembles that of α1-syntrophin (an rmsd value of 1.36 angstrom for the entire PDZ domains) (Tochio et al. (2000) J Mol Biol 295: 225-237).

WW3 Domain of Nedd4.

WW domains, named after two tryptophan residues highly conserved in the family, are protein interaction modules recognizing short proline-rich sequences (Bork and Sudol (1994) Trends Biochem Sci 19: 531-533). They are found in proteins with functions as diverse as cell cycle control, pre-mRNA 3′ end formation and targeted protein degradation (Sudol and Hunter (2000) Cell 103: 1001-1004; Lu et al. (1999) Science 283: 1325-1328; Morris et al (1999) J Biol Chem 274: 31583-31587; Morris and Greenleaf (2000) J Biol Chem 275: 39935-39943; Verdecia et al. (2000) Nat Struct Biol 7: 639-643). On the basis of ligand preferences, WW domains are segregated into at least five classes (Kasanov et al. (2001) Chem Biol 8: 231-241): Class I prefers peptide ligands with a core motif PPxY (Chen and Sudol (1995) Proc. Natl. Acad. Sci., USA, 92: 7819-7823); Class II—PPLP (Bedford et al. (1997) Embo J 16: 2376-2383); Class III—PxxGMxxPP (Bedford et al. Proc. Natl. Acad. Sci., USA, 95: 10602-10607); Class IV—(pS/pT)P (Lu et al. (1999) Science 283: 1325-1328); and Class V—RxPPGPPPxR (Komuro et al. (1999) J Biol Chem 274: 36513-36519).

The third WW domain of the mouse Nedd4 ubiquitin protein ligase (Nedd4-WW3) (Kumar et al. (1997) Genomics 40: 435-443) has been used as a target to screen a human brain cDNA library by TAIS. The peptides selected by the Nedd4-WW3 from the cDNA library, together with the names of the proteins from which they are derived, are shown in Table 5. The Nedd4-WW3 belongs to the Class I WW domains and a characteristic Class I core recognition motif PPxY is readily discernible in all selected peptide sequences (underlined in Table 5). In fact, if the selected peptides are subjected to unbiased analysis by software that is “unaware” of WW domain family ligand preferences and simply identifies homologous stretches in unrelated peptide sequences, the only common motif between four selected peptides is PPPY(E/D)EV (SEQ ID NO:64, Table 7).

TABLE 5

Results of screening of a human brain cDNA library with the third
WW domain of Nedd4 ubiquitin ligase as a target. Sequences and identities
of polypeptides selected by the Nedd4-WW3 domain. The PPxY core recognition
motif of WW domain family is underlined.

| | | SEQ |
|---|---|---|
| | | ID |
| Protein | Sequence | NO |
| >AF327246.1 | | |
| /gene = “SCN2A” | PPXYESL-WW3 | 65 |
| /product = “voltage- | STPEKTDMTPSTTSPPSYDSVTKPEKEKFEKDKSEKEDKGKDIRESKK | 66 |
| gated sodium | | |
| channel type II | | |
| alpha subunit” | | |
| /protein_id = “AAG | | |
| 53413.1” | | |
| >XM_001374 | P | |
| /gene = “LAPTM5” | LPxYxEA-WW2 ? | 67 |
| /product = “Lysosomal- | SSYRLIKCMNSVEEKRNSKMLQKVVLPSYEEALSLPSK- | 68 |
| associated | | |
| multispanning | PPxYESL-WW3 | 69 |
| membrane | -TPEGGPAPPPYSEV | 68 |
| protein-5” | | cont |
| /protein_id = “XP_{—} | | ′d |
| 001374.2” | | |
| >AF320999 | PPxYESL-WW3 | 70 |
| /gene = “Nogo-A” | 390_SAVPSAGASVIQPSSSPLEASSVNYESIKHEPENPPPYEEAMSVSLKK | 71 |
| /product = “Nogo-A | VSGIKEEIKEPENINAALQETEAPYISIACDLIKETKLSAEPAPDFSDYSEM | |
| protein short | AK-491 | |
| form” | | |
| /note = “alter- | | |
| natively spliced” | | |
| /protein_id = “AAG | | |
| 40878.1” | | |
| >AL137579.1/gene = | GPRTPHRVPGPWGPPEPLLLYRAAPPAYGRGGELHRGSLYRNGGQRGEGAGP | 72 |
| “DKFZp434A1010” | PPPYPTPSWSLHSEGQTRSYC> | |
| /note = “N- | | |
| chimaerin | | |
| homolog | | |
| F25965_3, | | |
| alternative | | |
| spliced” | | |
| /protein_id = “CAB | | |
| 70821.1” | | |

This motif is in good agreement with a recognition consensus for Nedd4-WW3, PPxYES(L/M) (SEQ ID NO:73), defined independently by artificial peptide repertoire analysis (Kay et al. (2000) FEBS Lett 480, 55-62). A contribution of peptide ligand residues C-terminal to the PPxY core to binding energy and specificity of interaction mediated by the Nedd4-WW3 domain has been demonstrated convincingly by the recently published solution structure of the Nedd4-WW3 domain complexed with the peptide derived from the β subunit of the epithelial sodium channel (EnaC), TLPIPGTPPPNYDSL (SEQ ID NO:74, Kanelis et al. (2001) Nat Struct Biol 8: 407-412). It should be noted that two PPxY motifs in the chimaerin homologue peptide, PPAYGRG (SEQ ID NO:75) and PPPYPTP (SEQ ID NO:73), do not conform well to the extended recognition consensus of Nedd4-WW3, PPxYES(L/M) (SEQ ID NO:76). A conceivable explanation is that they, or one of them, represent secondary recognition motif(s) for Nedd4-WW3 domain. Alternatively, the chimaerin homologue may be a false positive picked up due to avidity provided by two closely situated PPxY core motifs.

Nedd4 has been proposed to control stability and/or turnover of ENaC at the cell surface, presumably by directing its ubiquitination, which is followed by endocytosis and degradation of the channel (Staub et al. (1996) Embo J 15: 2371-2380; Staub et al. (1997) Embo J 16: 6325-6336; Abriel et al. (1999) J Clin Invest 103: 667-673). WW domains of Nedd4 are thought to function in this system as targeting modules, since they specifically bind subunits of ENaC. Deletions or point mutations in the PPxY motif on or y subunits of ENaC are associated with a hereditary form of hypertension, Liddle's syndrome, which is characterized by deregulated activity of ENaCs (Shimkets et al. (1994) Cell 79: 407-414. A number of authors have proposed that Nedd4 and Nedd4-like proteins, due to their unique structure comprising a membrane targeting C2 domain, two to four WW domains and a C-terminal HECT-type ubiquitin protein ligase domain, are strong candidates for regulators of ubiquitin-mediated turnover of many membrane proteins (Jolliffe et al. (2000) Biochem J 351 Pt 3, 557-565; Abriel et al. (2000) FEBS Lett 466: 377-380; Rotin et al. (2000) J Membr Biol 176: 1-17). Indeed, the yeast ubiquitin-protein ligase Rsp5p, a homologue of mammalian Nedd4 and Itch, is required for the ubiquitination and subsequent internalization of several plasma membrane proteins, including the alpha-factor receptor (Ste2p) (Hicke et al. (11996) Cell 84: 277-287; Dunn and Hicke (2001) Mol Biol Cell 12: 421-435), uracil permease (Galan et al. (1996) J Biol Chem 271: 10946-10952), general amino acid permease (Springael et al. (1998) Mol Biol Cell 9: 1253-1263) and others (Hicke (1997) Faseb J 11: 1215-1226). Therefore, it is reasonable to assume an existence of multiple Nedd4 targets in the cell.

Nogo-A, lysosomal-associated multispanning membrane protein 5 (LAPTM5), type II α subunit of voltage gated sodium channel (SCN2A) and a novel human protein with homology to chimaerin have been identified by TAIS as novel putative interaction partners of Nedd4 (Table 5). Notably, all but chimaerin homolog are membrane proteins.

Nogo-A

Nogo-A has been recently cloned independently by three different teams as a long sought myelin inhibitor of regenerating axons, and is the subject of intensive studies assessing the contribution of Nogo to the failure of axonal regeneration in the adult CNS (Prinjha et al. (2000) Nature 403: 383-384; GrandPre et al. (2000) Nature 403: 439-444; Chen et al. (2000) Nature 403: 434-439). A possible regulation of Nogo-A through ubiquitin-mediated degradation pathways may provide a fruitful framework for studies aiming to understand the molecular basis of CNS regeneration and plasticity.

LAPTM5

LAPTM5 was originally cloned as a lysosomal membrane associated protein that interacts with ubiquitin, developmentally downregulated and preferentially expressed in adult tissues with high cell turnover (Adra et al. (1996) Genomics 35: 328-337). The function of the protein is unknown. The rat homologue of mouse LAPTM5, Granule Cell Death-10 protein (GCD-10), is up-regulated in microglia in response to degeneration and cell death of neurons in vitro and in vivo and is involved in the dynamics of lysosomal membranes of activated microglia (Origasa et al. (2001) Brain Res Mol Brain Res 88: 1-13). To our knowledge, the present report is a first link that connects ubiquitin-dependent endocytic machinery to the integral lysosomal membrane protein, thus shedding light on the receiving end of this degradation pathway. Indeed, several authors have suggested a function for the Nedd4 yeast orthologue Rsp5p and its WW domains downstream of plasma membrane protein ubiquitination (Rotin et al. (2000) J Membr Biol 176: 1-17; Dunn and Hicke (2001) Mol Biol Cell 12: 421-435; Beck et al. (1999) J Cell Biol 146: 1227-1238). Recent report on localization of Rsp5p at multiple sites within endocytic pathways, such as plasma membrane invaginations, late endosomes and perivacuolar sites, supports the notion of a direct role for Rsp5p and ubiquitin in protein sorting and trafficking (Wang et al. (2001) Mol Cell Biol 21: 3564-3575). The ability of LAPTM5 to interact with both ubiquitin and Nedd4 suggests a potential role for LAPTM5 as a lysosomal receptor for ubiquitinated cargo destined for destruction.

SCN2A

The ability of neurons to communicate by generation and propagation of action potentials along their axons is crucially dependent on activity of voltage-gated sodium channels (VGSC) (Armstrong and Hille (1998) Neuron 20: 371-380). Identification of SCN2A as a putative interaction partner of Nedd4 ubiquitin ligase is indicative of a possible role of ubiquitin-mediated degradation pathways in the control of neuronal VGSC stability and/or turnover. In fact, a conservation of a PPxY motif, a presumptive WW domain binding site, within the C-termini of a number of sodium channels, was noticed as early as 1996 by Einbond, and Sudol (1996) FEBS Lett 384: 1-8. The functional significance of this conservation has been confirmed by experimental data indicating that both ENaC and the cardiac voltage-gated Na+ channel H1 (SCN5A) are regulated by Nedd4 ubiquitin-protein ligase in a WW domain dependent manner (Abriel et al. (2000) FEBS Lett 466: 377-380). Table 6 shows results of screening of a human brain cDNA library with the third WW domain of Nedd4 ubiquitin ligase as a target.

Table 6 shows results of screening of a human brain cDNA library with the third WW domain of Nedd4 ubiquitin ligase as a target. Homologous sequences shared by polypeptides selected with Nedd4-WW3 domain as defined by the BLOCK MAKER algorithm (see, e.g., http://www.blocks.fhcrc.org/blockmkr/make_blocks.html).

| | | |
|---|---|---|
| Sequence | ID | SEQ ID NO |

| | | | |
|---|---|---|---|
| 15 | PPSYDSV | SCN2A | 77 |
| 53 | PPPYPTP | N-chimaerin homolog | 78 |
| 46 | PPPYSEV | LAPTM5 | 79 |
| 35 | PPPYEEA | Nogo-A | 80 |
| | PPPYEEV | Consensus | 81 |
| | D | | |
| | PPxYESL | Kay et al. (2000) | 82 |

In Table 7 we show the C-termini of all proteins from the SWISS-PROT and TrEMBL databanks that share Nedd4 recognition site on the cardiac voltage-gated Na+ channel H1, PPSYDSV (SEQ ID NO:83).

Table 7 shows results of screening of a human brain cDNA library with the third WW domain of Nedd4 ubiquitin ligase as a target. C-terminal sequences of all proteins from SWISS-PROT and TrEMBL databases that share a PPSYDSV (SEQ ID NO:84), sequence (bold). Underlined are putative PEST sequences as defined by PESTfinder algorithm (http://www.at.embnet.org/embnet/tools/bio/PESTfind/). PPxYESL (SEQ ID NO:85, Kay et al. (2000) FEBS Lett 480: 55-62) and (P/L)PxYxEA (SEQ ID NO:86, Kasanov et al. (2001) Chem Biol 8: 231-241) recognition consensuses for Nedd4-WW3 and Nedd4-WW2 domains, respectively, as well as Nedd4-WW3 domain binding site on □EnaC (Kanelis et al. (2001) Nat Struct Biol 8: 407-412) are shown for comparison. VGSC—voltage gated sodium channel; CNS—central nervous system; PNS—peripheral nervous system. “>”—denotes carboxylate group.

| | | |
|---|---|---|
| | | Seq ID |
| Gene Name Accession | Origin No | NO |

| | |
|---|---|
| VGSCs from heart: | |
| PLGPPSSSSISSTSFPPSYDSVTRATSDNLQVRGSDYSHSEDLADFPPSPDRDRESIV | 87 |
| Q14524 | |
| SCN5A human | |
| RRSAPLSSSSISSTSFPPSYDSVTRATSDNLPVRASDYSRSEDLADFPPSPDRDRESIV | 88 |
| P15389 | |
| SCN5A rat | |
| RRSGPLSSSSISSTSFPPSYDSVTRATSDNLPVRASDYSRSEDLADFPPSPDRDRESIV | 89 |
| Q9JJV9 | |
| SCN5A mouse | |
| VGSCs from CNS: | |
| KLNENSTPEKTDMTPSTTSPPSYDSVTKPEKEKFEKDKSEKEDKGKDIRESKK | 90 |
| Q99250 | |
| SCN2A human | |
| KLNENSTPEKTDVTPSTTSPPSYDSVTKPEKEKFEKDKSEKEDKGKDIRESKK | 91 |
| P04775 | |
| SCN2A rat | |
| KLNGNSTPEKTDGSSSTTSPPSYDSVTKPDKEKFEKDKPEKESKGKEVRENQK | 92 |
| Q9NY46 | |
| SCN3A human | |
| KLNGNSTPEKTDGSSSTTSPPSYDSVTKPDKEKFEKDKPEKEIKGKEVRENQK | 93 |
| P08104 | |
| SCN3A rat | |
| VGSCs from PNS: | |
| DNVNSSSPEKTDATASTISPPSYDSVTKPDKEKYEKDKTEKEDKGKDGKETKK | 94 |
| Q28644 | |
| none rabbit | |
| VNENCALPDKSETASAASFPPSYDSVTRGLSDQINMSTSSSMQNEDEGTSKKVTAPGP | 95 |
| O46669 | |
| none dog | |
| FMANSGLPDKSETASATSFPPSYDSVTRGLSDRANINPSSSMQNEDEVAAKEGNSPGPQ | 96 |
| Q63554 | |
| SNS rat | |
| NVNENSSPEKTDVTASTISPPSYDSVTKPDQEKYETDKTEKEDKEKDESRK | 97 |
| O08562 | |
| none rat | |
| RLNGNSTTEKMDMTPSTASPPSYDSVTKPSKEKHEKDKSEREDKGKDVRHNRK | 98 |
| Q9YGN7 | |
| none newt | |
| Other VGSCs: | |
| NVNENSSPEKTDATASTISPPSYDSVTKPDQEKYETDKTEKEDKEKDESRK | 99 |
| Q62205 | |
| SCN9A mouse | |
| NVNENSSPEKTDATSSTTSPPSYDSVTKPDKEKYEQDRTEKEDKGKDSKESKK | 100 |
| Q15858 | |
| HNE-NA human | |
| ANDNGGLPDKSETASATSFPPSYDSVTRGLSDRANISTSSSMQNEDEVTAKEGKSPGPQ | 101 |
| Q62243 | |
| none mouse | |

As one can see, the PPSYDSV (SEQ ID NO:102) sequence: i) is strictly conserved across species and between different alpha subunit isoforms of cardiac and neuronal VGSCs; ii) is embedded in sequences shown to be prerequisite for proteins degraded through ubiquitin-directed endocytosis, such as PEST sequences, multiple serines and threonines (phosphorylation acceptors) and lysines (ubiquitination acceptors); and iii) conforms well to recognition consensus of the Nedd4 WW3 domain, PPxYES(L/M) (SEQ ID NO:103), defined recently by a combinatorial peptide library approach (Kay et al. (2000) FEBS Lett 480, 55-62). Remarkable parallels in the control of ENaC and cardiac sodium channel by Nedd4 ubiquitin-protein ligase (Abriel et al. (2000) FEBS Lett 466: 377-380), strict conservation of the Nedd4-WW3 recognition sequence within C-termini of cardiac and neuronal voltage gated sodium channels and an in vitro interaction of Nedd4-WW3 with a C-terminus of alpha subunit of neuronal VGSC (as noted in the present paper) strongly suggest a role of the Nedd4 ubiquitin-mediated endocytotic pathway in the regulation of stability and/or turnover of neuronal VGSC. It is relevant that high expression of Nedd4 was demonstrated in the heart and nervous tissues (Staub et al. (1996) Embo J 15: 2371-2380).

Chimaerin Homology

A novel protein homologous to human chimaerins has been identified by TAIS as a putative interaction partner of Nedd4. Homology to chimaerins is restricted to the first 85 out of 862 amino acids of the protein, which constitute a domain conserved in GTPase activators for Rho-like GTPases (RhoGAP domain). A role for Rho family GTPases has been demonstrated convincingly at different steps of endocytosis, intracellular sorting and trafficking, although the molecular mechanisms involved remain unknown (Ellis and Mellor (2000) Trends Cell Biol 10: 85-88; Chavrier and Goud (1999) Curr Opin Cell Biol 11: 466-475; Hall (1998) Science 279: 509-514; Ridley (1996) Curr Biol 6: 1256-1264). Interaction between the WW domain of Nedd4 and a chimaerin homolog may shed light on the mechanism of recruitment of Rho family GTPase machinery to the protein ligase complexes controlling ubiquitin-mediated endocytosis.

SH3 Domains.

The Src homology 3 (SH3) domain has become a prototype of protein interaction modules since it was first described as a conserved repeat in the N-terminus of Src family tyrosine kinases (Koch et al. (1991) Science 252: 668-674). Small, about 50-70 amino acids long, with a compact fold, SH3 domains recognize and bind peptide sequences with the core PxxP motif. The specificity of interaction within the SH3 family is determined by additional contacts formed between amino acids adjacent to the PxxP core of peptide ligand and variable amino acids within SH3 domain specificity pocket (Rickles et al. (1995) Proc. Natl. Acad. Sci., USA, 92: 10909-10913; Feng et al. (1995) Proc. Natl. Acad. Sci., USA, 92, 12408-12415). Peptide ligands can bind SH3 domains in two pseudosymmetrical (with respect to the PxxP core motif) orientations—the Class I orientation, ZxxPxxP, and the Class II orientation, PxxPxZ, where Z denotes the ligand residue(s) responsible for discrimination between individual SH3 domains (Feng et al. (1994) Science 266: 1241-1247).

The function of SH3 domains within the Src and Abl tyrosine kinases is believed to be two-fold. On one hand, through intramolecular interaction, SH3 domains of Src and Abl participate in the autoinhibitory control of the respective kinases (Sicheri and Kuriyan (1997) Curr Opin Struct Biol 7: 777-785; Barila and Superti-Furga (1988) Nat Genet 18: 280-282). On the other, they serve as targeting modules by binding to a specific subset of proteins containing polyproline sequences (Koch et al. (1991) Science 252: 668-674; Pawson and Nash (2000) Genes Dev 14: 1027-1047). Therefore, identification of binding partners of SH3 domains of the tyrosine kinases either directly suggests physiological targets of their activity or may indicate the multiprotein complexes to which they are targeted.

Crk is an adaptor protein composed of an SH2 domain and one or two (depending on the isoform) SH3 domains (Feller et al. (1998) J Cell Physiol 177: 535-552). By interacting with specific sets of proteins via their interaction modules, adaptor proteins function to provide a molecular connection between signal transduction pathways. Identification of interaction partners of an adaptor protein facilitates the unraveling of interconnections and possible cross-talk between different signaling cascades.

c-Src and c-Abl tyrosine kinases and the adaptor protein Crk are cellular counterparts of classical viral oncogenes, v-Src (Radke et al. (1980) Cell 21: 821-828), v-Abl (Rosenberg and Witte (1988) Adv Virus Res 35: 39-81) and v-Crk (Mayer et al. (1988) Nature 332: 272-275). The pathways affected by these oncogenes have been the subjects of extensive studies with a number proteins identified as interacting partners of the respective SH3 domains (Barfod et al. (1993) J Biol Chem 268: 26059-26062; Weng et al. (1994) Mol Cell Biol 14: 4509-4521; Kapeller et al. (1994) J Biol Chem 269: 1927-1933; Gout et al. (1993) Cell 75: 25-36; Weng et al. (1993) J Biol Chem 268: 14956-14963; Ren et al. (1993) Science 259: 1157-1161; Gertler et al. (1995) Genes Dev 9: 521-533; Ren et al. (1994) Genes Dev 8: 783-795; Knudsen et al. (1994) J Biol Chem 269: 32781-32787; Hasegawa et al. (1996) Mol Cell Biol 16: 1770-1776). Ligand preferences as well as the molecular basis of recognition specificity of Src-SH3, Abl-SH3 and Crk-SH3 domains have been recurrently addressed by screening of combinatorial peptide libraries and structural studies (Cheadle et al. (1994) J Biol Chem 269: 24034-24039; Rickles et al. (1994) Embo J 13: 5598-5604; Sparks et al. (1996) Proc. Natl. Acad. Sci., USA, 93: 1540-1544; Sparks et al. (1994) J Biol Chem 269: 23853-23856; Rickles et al. (1995) Proc. Natl. Acad. Sci., USA, 92: 10909-10913; Yu et al. (1994) Cell 76: 933-945; Feng et al. (1994) Science 266: 1241-1247; Musacchio et al. (1994) Nat Struct Biol 1: 546-551; Wu et al. (1995) Structure 3: 215-226).

We have identified by TAIS in non-exhaustive screens a number of previously described as well as novel putative interacting partners for Src, Abl and Crk SH3 domains (see Table 8).

TABLE 8
Summary of TAIS performed on a phage-displayed human
brain cDNA library with the indicated targets.

| | | Accession | Individual | | | |
|---|---|---|---|---|---|---|
| Target | Hits | (GenBank) | Hit Frequency | Novelty | Statistics | |
| rPSD95-PDZ | DGKζ | U51477 | 1 | Novel | From 11 clones | |
| (1 + 2 + 3) | | | | | analyzed: | |
| | | | | | Hits | 1 |
| | | | | | Frameshifts | 3 |
| | | | | | Untranslated region | 1 |
| | | | | | Undefined * | 6 |
| mNedd4-WW3 | Chimaerin homolog | AL137579 | 1 | Novel | From 7 clones | |
| | VGSC type II α | AF327246 | 2 (siblings) | Novel | analyzed: | |
| | LAPTM5 | XM_001374 | 1 | Novel | Hits | 7 |
| | Nogo-A | AF320999 | 3 (2 siblings + 1) | Novel | | |
| hSrc-SH3 | WIP | NM_003387 | 2 siblings | Novel | From 12 clones | |
| | dynamin | XM_011757 | 3 (2 siblings + 1) | ** | analyzed: | |
| | | | | | Hits | 5 |
| | | | | | Frameshifts | 5 |
| | | | | | Untranslated region | 2 |
| hAbl-SH3 | SNRPC | XM_004292 | 1 | Novel | From 27 clones | |
| | ZNF162 | XM_006534 | 1 | Novel | analyzed: | |
| | Aczonin/Piccolo | HSY19188 | 1 | Novel | Hits | 4 |
| | MEA11/MGEA6 | HSU73682 | 1 | Novel | Frameshifts | 12 |
| | | | | | Undefined | 11 |
| hCrk-SH3N | KIAA0716 | XM_004923 | 2 (siblings) | Novel | From 20 clones | |
| | DKFZp434KO31 | AL137317 | 11 | Novel | analyzed: | |
| | | | (3 independent | | Hits | 14 |
| | | | sibling groups: | | Frameshifts | 2 |
| | | | 7 + 2 + 2) | | Undefined | 4 |
| | DOCK1 | NM_001380 | 1 | *** | | |

* Undefined - see explanation in the text
** Gout et al. (1993) Cell 75: 25-36.
*** Hasegawa et al. (1996). Mol. Cell Biol. 16: 1770-1776.

In total, 77 clones that gave positive plaques on the membranes were analyzed by sequencing. 75 of them contained amino acid sequences that conformed to known recognition motifs of the respective target domains, thereby highlighting the performance of TAIS in the deliniation of target recognition preferences. The information about binding preferences such as recognition consensus can be used then for “in silico” identification of putative interactors of the respective target from protein databases (see example below).

In the screening experiments summarized above 40% of all positives clones displayed polypeptides that belong to known proteins demonstrating thus a high rate of true positives for direct in vitro identification of putative target interacting partners from cDNA libraries. Nucleotide sequences of 21 positive clones (27% of all analyzed) did not match any known protein coding sequences in NCBI database, though matches were found in the human EST database for all of them. Since a definite conclusion as to whether these sequences represent polypeptides from the human proteome or random peptides cannot be drawn at present, they have been designated as “undefined”. Given the statistics we expect that a significant fraction of undefined sequences represent novel uncharacterized proteins.

All peptides, except two, which were selected by tested SH3 domains, contained sequences that conformed to the described recognition consensuses of the respective SH3 domains (see, e.g., Table 9).

TABLE 9

Alignments of polypeptides selected from a
phage-displayed human brain cDNA library by
the indicated SH3 domains in comparison to
previously reported recognition consensuses
of corresponding SH3 domains. Underlined
residues in previously reported consensuses
for Src and Crk SH3 domains are position that
have been fixed in biased peptide libraries
used to define the respective consensuses.
ψ denotes aliphatic residues. Note the
additional specificity determinants
uncovered by TAIS for the Crk SH3 domain
at +4 and +5 positions (in respect to the
PxxP core) of the selected peptide ligands.

| | |
|---|---|
| | SEQ ID |
| ID | NO |

| | | |
|---|---|---|
| CRK-SH3 | | |
| VTSEPPALPPKPLAARSSH | KIAA0716 | 104 |
| SETISPLRPQRPKSQVMN | DOCK1 | 105 |
| APTSPPIVPLKSRHLVAAA | DKFZp434K031 | 106 |
| NLRGAPALPGRSLRPPVDAP | ICR1 | 107 |
| ELARSPSLPRKLRRLNEYYP | IICRK1 | 108 |
| SSQPRLPPKQRGNARAH | IICRK4 | 109 |
| MEKPCLPEKKKKKISQMW | IICRK6 | 110 |
| HGETPSLPKKKYKN | IICRK7 | 111 |
| PVIRPPLPPKVLGLQA | ICR8 | 112 |
| PxLPxKx+ | TAIS consensus | 113 |
| •P•LP•K* | | 114 |
| Src-SH3 | | |
| PRPIQSSLHNRGSPPVPG | WIP | 115 |
| RKRRPLPSPRLPPFPPSATREF | 6TAK | 116 |
| PPSPPTLARRTLPLSPAALKKNNN | 2BS | 117 |
| GPPPQVPSRPNRAPPGVPSRSGQA | dynamin | 118 |
| SSPPPRSLPTPPPRSLPTPP | 1BS | 119 |
| SGPRRAPRGLPPIPLRWGSERS | ITAK | 120 |
| PSxxPRxLPxxP | TAIS consensus | 121 |
| SLxxRPLPPLPP* | Other consensus | 122 |
| LxxRPLPx•P** | | 123 |
| RxLPPLP*** | | 124 |
| Ab1-SH3 | | |
| MPMMPGPPMMRPPARPMMVPTR | SNRPC | 125 |
| QHNPNGPPPPWMQPPPPPMNQGPHPP | ZNF162 | 126 |
| GASRDYFPPRDFPGPPPAPFAMR | MEA11 | 127 |
| IQAGGSRGPVRAPPTRPCPGASGTG | 1AS13 | 128 |
| RQSCEPWAGPRVAPPRPPGHQGSEGE | 2AA | 129 |
| WGRIYRGAPPTFAAPQAPKPFRQLLPM | 2AS13 | 130 |
| REGSCLQPLPPPPPPPRLRPVR | 3ABR | 131 |
| TKKPQREPPALPPPPPPLIKFL | 3AS13 | 132 |
| GGHRDPPKARPPRPPSAPKP | 4AB | 133 |
| EPLLPPPLPAPPAPPPVPA | 5AS | 134 |
| HRSSTMNPPPHTQPPSQPQPRPPIYS | 9AB | 135 |
| PPxxxPPxPP | TAIS Consensus | 136 |
| PPxΘxPPPΨP* | | 137 |
| PPPYPPPPIP | | 138 |

Θ is aromatic residue.
*Sparks et al. (1996) Proc. Natl. Acad. Sci., USA, 93: 1540-1544
**Rickles et al. (1995) Proc. Natl. Acad. Sci., USA, 92: 10909-10913
***Yu et al. (1994) Cell 76: 933-945

The case of the three SH3 domains is of a special interest, for it addresses a question of cross-reactivity between domains within the same family. Surprisingly enough, the analysis of 59 clones positive for interaction with the tested SH3 domains showed that SH3 domains from Crk, Src and Abl selected non-overlapping sets of polypeptides from the same library.

Previous studies of the Src SH3 domain family molecular recognition mechanism showed that specific amino acids of the peptide ligands that lie outside the SH3 core recognition motif play a critical role in ligand discrimination by related SH3 domains and contribute significantly to the affinity of interaction (Rickles et al. (1995) Proc. Natl. Acad. Sci., USA, 92: 10909-10913; Feng et al. (1995) Proc. Natl. Acad. Sci., USA, 92, 12408-12415). Careful inspection of the amino acid sequences of peptides selected by TAIS with SH3 domains of Src, Abl and Crk revealed that the vast majority of selected peptides contained at least one continuous stretch featuring additional specificity determinants outside the SH3 core recognition sequence. Some of these determinants have been described previously, whereas others appear to be novel (see Table 9).

As in the case of the Nedd4-WW3 domain, it was possible to built up an extended recognition consensus for Crk SH3 domain without a priori knowledge about SH3 domain recognition preferences. This fact suggests that the Crk-SH3 domain has a strong preference for one of the two possible pseudosymmetrical orientations described for SH3 ligands, namely the Class II orientation, and exhibits a strong affinity for its cognate ligands. The majority of peptides selected by Crk-SH3 domain contained positively charged residues at position +4 and/or +5 following their PxxP cores. These residues may represent additional specificity determinant(s) not previously reported. The presence of multiple SH3 core motifs in both orientations within the same selected polypeptides prevented unambigous mapping of Src and Abl SH3 domain binding sites without knowledge of their recognition motifs.

Collectively, the results of screens performed with PDZ, WW and SH3 domains suggest that the TAIS format allows detection of interactions in a physiologically relevant range of affinities and is well suited for the characterization of ligand preferences of protein interaction modules.

Conclusions

A significant fraction of all specific protein-protein associations in the cell may be mediated by specialized peptide recognition domains such as PDZ, SH3, WW, EH, SH2, etc. Indeed, 3300 proteins out of 6148 predicted ORFs in the yeast proteome have been reported to contain the SH3 domain recognition core PxxP (Zucconi et al. (2000) FEBS Lett 480: 49-54; Cherry et al. (1998) Nucleic Acids Res 26: 73-79). Similarly, SH3 and PDZ domains were ranked as 14^{th} and 19^{th}, respectively, among the most populous domain families in the human proteome (Lander et al. (2001) Nature 409: 860-921). On the qualitative side, protein interaction modules, in the context of proteins with enzymatic, scaffolding or adaptor activity, are often constituents of a node of a protein interaction network, mediating multiple connections that diverge from or converge onto the node. Therefore, the identification of interacting partners of peptide interaction modules would contribute significantly to assembly of a comprehensive protein interaction map.

We have developed a new in vitro method, TAIS, that allows rapid screening of cDNA libraries for binding partners of peptide interaction modules. PDZ, WW and SH3 domains from PSD95, Nedd4, Abl, Crk and Src proteins were tested as targets. Summaries and statistics of test screens are compiled in Table 1. Two known and 12 novel potential interacting partners of these well studied domains were identified from a human brain cDNA library. All novel putative interacting partners contained recognition sequences of the respective target domains. Moreover, the absence of cross-reactivity between domains from the same family (SH3) and the presence of conserved ligand residues outside the family cores in all tested cases indicate high selectivity of the novel screening format. Most of the interactions make good sense in terms of biological relevance in the context of the known functions of PSD95, Nedd4, Src and Abl proteins, and allow generation of testable hypotheses about the functionality of detected interactions.

Deciphering rules that dictate binding specificity of protein interaction modules, or “protein recognition code,” (Cherry et al. (1998) Nucleic Acids Res 26: 73-79; Sudol (1998) Oncogene 17: 1469-1474) would greatly facilitate mapping of protein-protein interactions on a genomic scale by bioinformatic tools. In this regard, TAIS of cDNA libraries is a powerful complement to traditional random peptide library analysis. Indeed, we have confirmed known recognition consensuses for all protein interaction modules tested, defined a recognition consensus for the tandem of the first two PDZ domains of PSD95, and identified additional putative specificity determinants for the Crk-SH3 domain.

Experimental Protocol

GST Fusions.

GST fusion constructs of, PDZ domains from rat PSD95 protein, human Src, Abl and Crk SH3 domains were kindly provided by Brian Kay, University of Wisconsin-Madison. The third WW domain from mouse Nedd4 was amplified by PCR from Nedd4 cDNA supplied by Sharad Kumar, Hanson Center for Cancer Research, Adelaide, and cloned into the pGEX-2TK expression vector. All constructs were verified by sequencing.

Target Protein Preparation.

Immobilized GST fusions of target proteins were purified according to the supplier's instructions (Pharmacia Biotech.). To prepare biotinylated target complexes with streptavidin-alkaline phosphatase (STRAP) conjugate in solution, target domains were released from Glutathione Sepharose 4B beads by thrombin cleavage and mixed with freshly prepared water solution of EZ-link™ Sulfo-NHS-LC-LC-biotin (Pierce) at a molar ratio of 1:5. Biotinylation reaction was incubated for 30 minutes at room temperature followed by purification on MicroSpin G-25 column (Pharmacia Biotech.). The extent of biotinylation was kept at 1 to 2 moieties of biotin per target molecule. For detection of positive plaques on membranes, 5 μg of biotinylated target per membrane were pre-mixed with STRAP conjugate at a molar ratio of 4:1 to ensure multivalent target presentation and incubated for 10 minutes at RT before use in Tris-buffered saline, pH7.4+0.1% Tween 20 (TBS-T).

TAIS Protocol.

30 μg of target GST fusion immobilized on sepharose beads was blocked in 1 ml of 0.5% bovine serum albumin (BSA) in TBS-T for 1 hour at RT on a tumbler. After 3×1 ml washes with TBS-T, beads were mixed with an aliquot of cDNA library (10⁸ pfu) (Novagen) in 1 ml of 0.5% BSA in TBS-T and incubated at RT for 90 minutes on a tumbler. After 5×1 ml washes with TBS-T, the phages bound to the target were eluted by incubation of washed beads in 200 μl of 1% SDS for 15 minutes at RT. 2 equal parts of eluate were plated on two 150 mm agar plates with BLT5615 host (Novagen). Plates were incubated at 37° C. to develop plaques, usually for 2 to 3 hours. Plates with developed plaques were pre-cooled for 45 minutes at 4° C. and overlaid with 132 mm nitrocellulose membranes (Schleicher&Schuell) for 10 minutes. While on plates, membranes were punctured on periphery asymmetrically with red hot needles to introduce a coordinate system. After plaque lift membranes were blocked with 1% BSA in TBS for 1 hour at RT and left overnight at 4° C. on a rocker with 25 ml of 0.5% BSA in TBS-T containing 5 μg of biotinylated target complexed to STRAP. After extensive washing with TBS-T, positive plaques on membranes were developed with insoluble AP substrate, BCIP/NBT (Sigma). Individual positive plaques were identified on plates and picked up for sequencing. If density of plaques was too high to pick up individual phage, agar stubs containing positive plaques were excised and phages from stubs eluted in PBS. Eluted phages were plated for a secondary screening on membranes. T7 phage DNA was prepared for sequencing with lambda DNA Wizard kit from Promega.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of identifying interacting proteins from a plurality of potentially interacting proteins, said method comprising:
i) contacting one or more target proteins with a protein display library comprising a plurality of potential binding proteins for said one or more target proteins;
ii) selecting members of said protein display library that bind to said one or more target proteins to provide a preselected set of potential binding proteins;
iii) separating said members of said preselected set of potential binding proteins from the bound target protein and immobilizing said members on a solid support such that said members are spatially addressable; and
iv) contacting members of said preselected set of potential binding proteins with one or more target proteins; and
v) detecting specific binding of members of said preselected set of potential binding proteins with said one or more target proteins whereby binding of a member of said set of potential binding partners with a target protein indicates that said member and said target protein are interacting proteins.

2. The method of claim 1, wherein said one or more target proteins are attached to a solid support.

3. The method of claim 1, wherein said protein display library is a phage- or bacterial-display library.

4. The method of claim 3, wherein said phage- or bacterial-display library is a phage display library.

5. The method of claim 4, wherein said phage display library is a lytic phage library.

6. The method of claim 1, wherein said separating comprises amplifying members of said protein display library that bind to said one or more target proteins.

7. The method of claim 1, wherein said separating and/or immobilizing comprises amplifying members of said protein display library that bind to said one or more target proteins.

8. The method of claim 7, wherein said amplifying comprises amplification of said members when they are spatially separated and addressable.

9. The method of claim 3, wherein said phage- or bacterial-displayed library comprises a cDNA library.

10. The method of claim 1, wherein said protein display library comprises at least 100 different members.

11. The method of claim 10, wherein said protein display library comprises at least 1000 different members.

12. The method of claim 2, wherein said selecting comprises removing unbound members of said protein display library from said solid support.

13. The method of claim 1, wherein said selecting comprises capturing said one or more target proteins using an affinity matrix.

14. The method of claim 1, wherein contacting members of said preselected set of potential binding partners with one or more target proteins comprises adsorbing members of said preselected set of potential binding partners to a solid support.

15. The method of claim 14, wherein said solid support is a membrane.

16. The method of claim 1, wherein said detecting comprises detecting a label attached to said target protein.

17. The method of claim 16, wherein said label is selected from the group consisting of a fluorescent label, a radioactive label, an enzymatic label, a colorimetric label, and a magnetic label.

18. The method of claim 1, wherein:
said contacting of step (i) comprises contacting said one or more target proteins with a protein display library where said one or more target proteins are attached to a solid support;
said contacting of step (iv) comprises attaching members of said preselected set of potential binding proteins to a solid support to provide a set of attached preselected potential binding proteins and contacting the attached preselected potential binding proteins with the one or more target proteins.

19. The method of claim 18, where the one or more target proteins used in the contacting of step (iv) are labeled with a detectable label before the target proteins are contacted to the preselected potential binding proteins.

20. The method of claim 18, where the one or more target proteins used in the contacting of step (iv) are labeled with a detectable label simultaneous with or after the target proteins are contacted to the preselected potential binding proteins.

21. The method of claim 18, further comprising sequencing the nucleic acid encoding the displayed protein on a member of the preselected display library that binds to the target protein.

22. The method of claim 1, wherein:
said contacting of step (i) comprises contacting said one or more target proteins with a protein display library where said one or more target proteins and said protein display library are in solution.

23. The method of claim 22, wherein said selecting comprises capturing target proteins bound to members of said protein display library using an affinity matrix that specifically binds the target proteins or a tag attached to the target proteins.

24. The method of claim 23, wherein said contacting of step (iv) comprises attaching members of said preselected set of potential binding proteins to a solid support to provide a set of attached preselected potential binding proteins and contacting the attached preselected potential binding proteins with the one or more target proteins.

25. The method of claim 24, where the one or more target proteins used in the contacting of step (iv) are labeled with a detectable label before the target proteins are contacted to the preselected potential binding proteins.

26. The method of claim 24, where the one or more target proteins used in the contacting of step (iv) are labeled with a detectable label simultaneous with or after the target proteins are contacted to the preselected potential binding proteins.

27. The method of claim 1, wherein, said detecting comprises determining the amino acid sequence of a member of said set of potential binding partners that binds a target protein.

28. The method of claim 1, further comprising recording the amino acid sequence or identity of a member of said set of potential binding partners that binds a target protein in a database of proteins that interact with the target.

29. A method of identifying proteins or nucleic acids that interact with target moieties from a nucleic acid or protein library comprising a plurality of nucleic acids or proteins, said method comprising:
i) contacting one or more target moieties with said library;
ii) selecting members of said library that bind to said one or more target moieties to provide a preselected set of potential binding partners;
iii) separating said members of said preselected set of potential binding partners from the bound target and immobilizing said members on a solid support such that said members are spatially addressable;
iv) contacting members of said preselected set of potential binding partners with one or more target moieties; and
v) detecting binding of members of said set of potential binding partners with said one or more target moieties whereby binding of a member of said set of potential binding partners with a target binding moiety indicates that said member is a binding partner that interacts with the target moiety.

30. The method of claim 26, wherein said library is selected from the group consisting of a phage display library, a bacterial display library, a yeast display library, a eukaryotic virus library, a direct encoded plasmid library.

31. The method of claim 26, wherein said library is an in vitro display library selected from the group consisting of a covalent display technology (CDT) library, a polysome display library, and an RNA-peptide fusion library.

32. A method of identifying proteins that interact with target moieties from a plurality of potentially interacting proteins, said method comprising:
i) contacting one or more target moieties with a protein display library comprising a plurality of potential binding partners for said target moieties;
ii) selecting members of said protein display library that bind to said one or more target moieties to provide a preselected set of potential binding partners;
iii) separating said members of said preselected set of potential binding proteins from the bound target protein and immobilizing said members on a solid support such that said members are spatially addressable; and
iv) contacting members of said preselected set of potential binding partners with one or more target moieties; and
v) detecting binding of members of said set of potential binding partners with said one or more target moieties whereby binding of a member of said set of potential binding partners with a target binding moiety indicates that said member is a protein that interacts with the target moiety.

33. The method of claim 32, wherein said target moiety is selected from the group consisting of a nucleic acid, a lipid, a carbohydrate, a glycoprotein, a small organic molecule, and an inorganic molecule.

34. The method of claim 32, wherein said target moiety is a DNA or an RNA.

35. A kit for identifying interacting proteins from a plurality of potentially interacting proteins, said kit comprising:
a protein display library; and
instructional materials providing protocols for the method of claim 1.

36. The kit of claim 35, wherein said protein display library is a bacterial or phage display library.

37. The kit of claim 36, wherein said bacterial or phage display library comprises a cDNA library.
